SEQUENCE LISTING (16 sequences)

SEQ ID NO: 1 - DNA, 20 bp, synthetic construct
  source 1..20: mol_type = other DNA
cctcacttcactgcactgta

SEQ ID NO: 2 - DNA, 20 bp, synthetic construct
  source 1..20: mol_type = other DNA
ccgccgtccaattttaatca

SEQ ID NO: 3 - DNA, 21 bp, synthetic construct
  source 1..21: mol_type = other DNA
cttcaggaggtggtggtagca

SEQ ID NO: 4 - DNA, 22 bp, synthetic construct
  source 1..22: mol_type = other DNA
aaccaagtttgagacggaacag

SEQ ID NO: 5 - DNA, 21 bp, synthetic construct
  source 1..21: mol_type = other DNA
gatcgccacctacaggaagct

SEQ ID NO: 6 - DNA, 25 bp, synthetic construct
  source 1..25: mol_type = other DNA
gagtatgaggccctgctgaacatca

SEQ ID NO: 7 - DNA, 24 bp, synthetic construct
  source 1..24: mol_type = other DNA
atctctgagatgaaccggatgatc

SEQ ID NO: 8 - DNA, 23 bp, synthetic construct
  source 1..23: mol_type = other DNA
ggcctgctgagatcaaagactac

SEQ ID NO: 9 - DNA, 20 bp, synthetic construct
  source 1..20: mol_type = other DNA
  modified_base 15: mod_base = OTHER; note = 5-formyluracil
caggttttctttccntagct

SEQ ID NO: 10 - DNA, 17 bp, synthetic construct
  source 1..17: mol_type = other DNA
cgtcggcccaggacttg

SEQ ID NO: 11 - DNA, 23 bp, synthetic construct
  source 1..23: mol_type = other DNA
cacctgtccatccactgactctt

SEQ ID NO: 12 - DNA, 19 bp, synthetic construct
  source 1..19: mol_type = other DNA
gagcggaatccacctccac

SEQ ID NO: 13 - DNA, 23 bp, synthetic construct
  source 1..23: mol_type = other DNA
actcatgttctgcatcccagact

SEQ ID NO: 14 - DNA, 21 bp, synthetic construct
  source 1..21: mol_type = other DNA
gcgggtggtggtcttttggat

SEQ ID NO: 15 - DNA, 21 bp, synthetic construct
  source 1..21: mol_type = other DNA
cagattggcgcactgtttctt

SEQ ID NO: 16 - DNA, 24 bp, synthetic construct
  source 1..24: mol_type = other DNA
cactgtggctgtgagaatcttgtt

(19) United States
(12) Patent Application Publication
(10) Pub. No.: US 2023/0414674 A1
(43) Pub. Date: Dec. 28, 2023

(54) METHODS AND COMPOSITIONS FOR HAIR FOLLICLE GENERATION

(71) Applicant: Sanford Burnham Prebys Medical Discovery Institute, La Jolla, CA (US)

(72) Inventors: Evan Y. SNYDER, La Jolla, CA (US); Michel R. IBRAHIM, Minia (EG)

(21) Appl. No.: 18/325,745

(22) Filed: May 30, 2023

Related U.S. Application Data
(60) Provisional application No. 63/347,501, filed on May 31, 2022.

(51) Int. Cl. A61K 35/36; A61P 17/14; C12N 5/071; A61K 9/00

(52) U.S. Cl. CPC A61K 35/36; A61P 17/14; C12N 5/0628; A61K 9/0021

(57) ABSTRACT

Described herein are compositions and methods useful for hair follicle generation comprising transplanting human pluripotent stem cell-derived hair follicle bulge stem cells, wherein the developmental and molecular requirements for the generation of hair follicle following transplantation is ensured.

### CROSS-REFERENCE

This application claims the benefit of U.S. Provisional Application No. 63/347,501, filed May 31, 2022, which is incorporated herein by reference in its entirety.

### SEQUENCE LISTING

The instant application contains a Sequence Listing which has been submitted electronically in XML file format and is hereby incorporated by reference in its entirety. Said XML copy, created on Sep. 8, 2023, is named 42256-613_201_SL.xml and is 15,688 bytes in size.

### BACKGROUND

Follicular neogenesis is the generation of new hair follicles (HF) after birth. Although humans are born with a full complement of hair follicles, hair follicles can change in size and growth characteristics. For example, hair follicles can ultimately degenerate and disappear as in baldness or in permanent scarring (cicatricial) alopecias. Other common baldness and less common hair loss conditions, such as discoid lupus erythematosis, congenital hypotrichosis, lichen planopilaris, and other scarring alopecias are in need of regeneration of HF.

### SUMMARY

In certain aspects, disclosed herein is a method of growing a hair follicle comprising: (a) preparing human induced pluripotent stem cells (hiPSCs); (b) differentiating the hiPSCs into hair follicle bulge stem cells (HFBSCs); and (c) implanting the HFBSCs into skin of a subject.

In some embodiments, the hiPSCs have one, two, three, four, five, six, or more markers selected from the group consisting of CD200, ITGA6, ITGB1, OCT4, NANOG, SOX2, TRA-1-60, TRA-1-81 and SSEA4. In some embodiments, the HFBSCs have one, two, three, four, five, six, or seven markers selected from the group consisting of CD200, ITGA6, ITGB1, KRT15, KRT18, KRT19, and P63. In some embodiments, the preparing in (a) comprises introducing, by electroporation, non-integrating episomal plasmid vectors encoding OCT3/4, SOX2, KLF4, L-MYC, LIN28 and an shRNA for human p531. In some embodiments, the electroporation is via a Neon transfection system. In some embodiments, the differentiating in (b) comprises formation of embryoid bodies (EBs) in a floating culture. In some embodiments, the differentiating in (b) comprises plating the EBs onto coated plates. In some embodiments, the coated plates are collagen I coated plates. In some embodiments, the method further comprises, prior to (c), (b1) differentiating the hiPSCs into keratinocytes. In some embodiments, the differentiating in (b1) comprises employing all-trans retinoic acid (ATRA) and L-ascorbic acid (L-AA) to induce the hiPSC to form ectoderm and then the addition of bone morphogenic protein-4 (BMP-4) and epidermal growth factor (EGF). In some embodiments, the differentiating in (b1) is according to a sequential differentiation protocol. In some embodiments, the implanting in (c) comprises intradermal injection. In some embodiments, the implanting in (c) occurs at 15-19 days in vitro (DIV). In some embodiments, the implanting in (c) occurs 16-18 DIV. In some embodiments, the HFBSCs have not yet started expressing the keratinocyte associated molecules KRT5 and KRT14. In some embodiments, the method further comprises treating hair loss and/or a condition in a subject in need thereof, the condition is alopecia, ectodermal dysplasia, monilethrix, Netherton syndrome, Menkes disease, or hereditary epidermolysis bullosa. In some embodiments, the subject is a human subject.

In certain aspects, disclosed herein is a composition, comprising (a) hair follicle bulge stem cells (HFBSCs); and (b) media, wherein the HFBSCs express markers CD200, ITGA6, ITGB1, KRT15, KRT18, KRT19, and P63.

In some embodiments, the HFBSCs do not express the keratinocyte associated molecules KRT5 or KRT14. In some embodiments, the composition is made by a process comprising: (a) preparing human induced pluripotent stem cells (hiPSCs); and (b) differentiating the hiPSCs into the HFBSCs. In some embodiments, the hiPSCs have one, two, three, four, five, six, or more markers selected from the group consisting of CD200, ITGA6, ITGB1, OCT4, NANOG, SOX2, TRA-1-60, TRA-1-81 and SSEA4. In some embodiments, the preparing hiPSCs in (a) comprises introducing, by electroporation, non-integrating episomal plasmid vectors encoding OCT3/4, SOX2, KLF4, L-MYC, LIN28 and an shRNA for human p531. In some embodiments, the differentiating in (b) comprises formation of embryoid bodies (EBs) in a floating culture. In some embodiments, the differentiating in (b) comprises plating the EBs onto coated plates. In some embodiments, the coated plates are collagen I coated plates. In some embodiments, the HFBSCs is made by the method disclosed herein.

In certain aspects, disclosed herein is a hair follicle replacement method, comprising: (a) obtaining human pluripotent stem cells (hPSCs); (b) differentiating the hPSCs, thereby producing differentiated hPSCs toward becoming keratinocytes; (c) capturing and isolating at least a portion of the differentiated hPSCs, wherein the portion of the differentiated hPSCs expresses hair follicle bulge stem cell markers (HFBSCM); and (d) transplanting the portion of the differentiated hPSCs into a patient in need thereof.

In some embodiments, the hPSCs are human induced pluripotent stem cells (hiPSCs). In some embodiments, the hPSCs are hiPSC-derived hair follicle bulge stem cells (hiPSC-HFBSC). In some embodiments, the hiPSCs are derived from cells of the patient. In some embodiments, the cells of the patient are selected from the group consisting of fibroblasts, renal epithelial cells, and blood cells. In some embodiments, the transplanting in (d) occurs at least 15 days after the differentiating in (b). In some embodiments, the transplanting in (d) occurs at least 16 days after the differentiating in (b). In some embodiments, the transplanting in (d) occurs at least 17 days after the differentiating in (b). In some embodiments, the transplanting in (d) occurs at least 18 days after the differentiating in (b). In some embodiments, the transplanting in (d) occurs at least 19 days after the differentiating in (b). In some embodiments, the portion of the differentiated hPSCs is at a stage before the portion of the differentiated hPSCs has experienced downregulation of key integrins and key surface glycoproteins and before the portion of the differentiated hPSCs has started expressing keratinocyte-associated molecules. In some embodiments, the key integrins comprise at least integrin α6 and/or integrin β1. In some embodiments, the key surface glycoproteins comprise at least CD200. In some embodiments, the stage is before the portion of the differentiated hPSCs has started expressing keratinocyte-associated molecules KRT5 and KRT14. In some embodiments, the stage is after the portion of the differentiated hPSCs has expressed at least one of KRT15, KRT18, and KRT19, and P63. In some embodiments, the transplanting in (d) comprises transplanting the portion of the differentiated hPSCs intradermally above the muscle coat. In some embodiments, the capturing and isolating in (c) comprises flow cytometry. In some embodiments, the capturing and isolating in (c) comprises capturing and isolating cells that co-express CD200 and integrin α6. In some embodiments, the capturing and isolating in (c) comprises capturing and isolating cells that co-express CD200 and integrin β1. In some embodiments, the patient in need thereof has alopecia or has lost hair follicles from an injury or burn.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The patent application contains at least one drawing executed in color. Copies of this patent or patent application with color drawing(s) will be provided by the Office upon request and payment of the necessary fee.

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 depicts protocol developed to generate HFBSCs and keratinocytes from human induced pluripotent stem cells (hiPSCs).

FIG. 2 shows co-expression of CD200, ITGA6, and ITGB1 along with cardinal pluripotency markers on human embryonic stem cells (hESCs) and hiPSCs. Panels A-C show immuno-cytochemical (ICC) analysis of CD200, ITGA6, and ITGB1, each with NANOG, respectively. Panels D-F show flow cytometric (FC) analysis of CD200 with TRA-1-60, ITGA6 with SSEA4, and ITGB1 and TRA-1-60, respectively.

FIG. 3 displays immunocytochemical characterization of hiPSC-HFBSCs, as generated by the protocol in FIG. 1. Panels A-G display immunoreaction of hiPSC-HFBSCs for (Panel A) ITGA6 and KRT18, (Panel B) ITGA6 and P63, (Panel C) ITGA6 and KRT15, (Panel D) ITGB1 and KRT18, (Panel E) ITGB1 and P63, (Panel F) ITGB1 and KRT15, and (Panel G) KRT19 and P63, respectively.

FIG. 4 depicts analysis of the dynamics of the relative temporal expression of molecules associated with pluripotency (OCT4), with HFBSC (P63, KRT15, KRT19, KRT8, KRT18), and with keratinocytes (KRT5, KRT14) in hiPSC-derived cells at various days of the differentiation protocol in FIG. 1.

FIG. 5. shows the characterization of the hiPSC-derived keratinocytes in relation to the hiPSC-HFBSCs which emerge earlier. Panels A-B show flow cytometric (FC) analysis of the co-expression of (Panel A) CD200 and ITGA6 and (Panel B) CD200 and ITGB1 on hiPSCs on DIV 0-25, respectively. Panel C shows the immunocytochemical analysis of the expression of KRT14 at DIV 25 of the differentiation protocol.

FIG. 6 displays the co-culture of hiPSC-HFBSCs and MDCs.

FIG. 7 depicts the histologic evaluation of donor-derived HFs following intradermal transplantation of hiPSCs-HFBSCs. Panel A depicts a representative HF and epidermal cyst lined by multilayered epidermis. Panel B depicts the positive immunoreactivity of the HF in Panel A for HSNA. Panel C depicts an epidermal cyst showing multilayered epidermis with multiple HFs radiating from it. Panel D depicts the positive immunoreactivity of the HF in Panel C for HSNA. Panel E depicts the positive immunostaining of a representative reconstituted HF using antibody against KRT12. Panels F and G depict positive immunoperoxidase staining of representative reconstituted HFs with an antibody against TDAG51. Panel H depicts the immunopositive results for HSCA for the reconstituted epidermis.

FIG. 8 shows the characterization of hiPSCs derived from primary human fibroblasts. Panel A shows morphology of hiPSC colonies. Panel B shows immunocytochemical analysis: hiPSC clones express markers definitive of pluripotent cells, TRA-1-60, NANOG and DAPI. Panel C shows immunocytochemical analysis: hiPSC clones express markers definitive of pluripotent cells, TRA-1-81, SOX2 and DAPI. Panel D shows immunocytochemical analysis: hiPSC clones express markers definitive of pluripotent cells, SEAA4, OCT4 and DAPI. Panel E shows flow cytometry of the hiPSC clones express markers indicative of the pluripotent state, TRA-1-81 and SOX2. Panel F shows flow cytometry of the hiPSC clones express markers indicative of the pluripotent state, SSEA4 and OCT4.

FIG. 9A displays co-expression of CD200 along with SOX2 and DAPI on hESCs and on hiPSCs using immunocytochemistry. FIG. 9B displays co-expression of ITGA6 along with SOX2 and DAPI on hESCs and on hiPSCs using immunocytochemistry. FIG. 9C displays co-expression of ITGB1 along with SOX2 and DAPI on hESCs and on hiPSCs using immunocytochemistry.

FIG. 10A depicts flow cytometrical (FC) analysis of markers on hPSC's co-expression of CD200 and TRA-1-81 by hPSC. FIG. 10B depicts flow cytometrical (FC) analysis of markers on hPSC's co-expression of CD200 and SSEA4 by hPSC. FIG. 10C depicts flow cytometrical (FC) analysis of markers on hPSC's co-expression of ITGB1 and TRA-1-81 by hPSC. FIG. 10D depicts flow cytometrical (FC) analysis of down-regulation of pluripotency-associated markers TRA-1-81 on hiPSC-HFBSC at different stages of differentiation. FIG. 10E depicts flow cytometrical (FC) analysis of down-regulation of pluripotency-associated markers OCT4 and SSEA4 at different stages of differentiation.

FIGS. 11A-11C show human origin of the donor-derived reconstituted HFs by showing the positive immunoreactivity for human specific nuclear antigen (HSNA)(green). FIG. 11D shows human origin of the donor-derived reconstituted HFs by showing the positive immunoreactivity for human specific cytoplasmic antigen (HSCA) (green) in the reconstituted epidermis and HF.

FIG. 12A displays that a primary human HF can serve as a positive control for human specific nuclear antigen (HSNA) (green). FIG. 12B displays that a primary human HF can serve as a positive control for human specific cytoplasmic antigen (HSCA) (green). FIG. 12C displays that a primary human HF can serve as a positive control for KRT15 (green). FIG. 12D displays that a primary human HF can serve as a positive control for TDAG51.

### DETAILED DESCRIPTION

As disclosed herein, when using human induced pluripotent stem cells (hiPSCs) to achieve hair follicle (HF) replacement, one can emulate the earliest fundamental developmental processes of gastrulation, ectodermal lineage commitment, and dermo-genesis. Viewing hiPSCs as a model of the epiblast, mapping the dynamic up- and down-regulation of the developmental molecules that determine HF lineage can provide insights to ascertain the precise differentiation stage and molecular requirements for grafting HF-generating progenitors. To yield an integrin-dependent lineage like the HF in vivo, hiPSC derivatives may need to be co-expressed, just prior to transplantation, the following combination of markers: integrins a6 and b1 and the glycoprotein CD200 on their surface; and, intracellularly, the epithelial marker keratin 18 and the hair follicle bulge stem cell (HFBSC)-defining molecules transcription factor P63 and the keratins 15 and 19. If the degree of trichogenic responsiveness indicated by the presence of these molecules is not achieved (they peak on Days 11-18 of the protocol), HF generation may not be possible. Conversely, if differentiation of the cells is allowed to proceed beyond the transient intermediate progenitor state represented by the HFBSC, and instead cascades to their becoming keratin 14 keratin 5 CD200− keratinocytes (Day 25), HF generation is equally impossible. Day 16-18 of differentiation may be the preferred time for transplanting. At this time point, the hiPSCs have lost pluripotency, have attained optimal expression of HFBSC markers, have not yet experienced downregulation of key integrins and surface glycoproteins, have not yet started expressing keratinocyte-associated molecules, and have sufficient proliferative capacity to allow a well-populated graft. This panel of markers may be used for isolating (by cytometry) HF-generating derivatives away from cell types unsuited for this therapy as well as for identifying trichogenic drugs.

Regenerative Medicine seeks to use stem cells to replace cells that have undergone destruction or senescence and death. Among the most sought cell types are hair follicles (HFs) for patients with inherited or immunogenic alopecia, or alopecia following severe wounding (e.g., burns, trauma, or surgery), or androgenetic alopecia (Chueh, S. C., et al. Therapeutic strategy for hair regeneration: hair cycle activation, niche environment modulation, wound-induced follicle neogenesis, and stem cell engineering. Expert Opin Biol Ther. 2013; 13(3):377-391). There has long been a debate over what qualities a stem cell and its derivatives should possess to enable HF replacement (see, e.g., Veraitch, O., et al. Human induced pluripotent stem cell-derived ectodermal precursor cells contribute to hair follicle morphogenesis in vivo. J Invest Dermatol. 2013; 133(6):1479-1488; and Yang, R., et al. Generation of folliculogenic human epithelial stem cells from induced pluripotent stem cells. Nat Commun. 2014; 5:3071). Herein presented the developmental and molecular requirements that human pluripotent stem cells (hPSCs) and their derivatives must acquire to reconstitute HFs, whether using human embryonic stem cells (hESCs) or, more immunologically desirable, patient recipient-specific human induced pluripotent stem cells (hiPSCs). These insights into successful engraftment were gained by examining the dynamics of up- and down-regulation of the key molecules that ensure proper HF lineage commitment by hPSCs. hPSCs intended for HF replacement may need to co-express the following combination of markers: key components of the integrin signaling pathway-integrin a6 (ITGA6) (see, e.g., Li, A. et al. Identification and isolation of candidate human keratinocyte stem cells based on cell surface phenotype. Proc Natl Acad Sci USA. 1998; 95(7):3902-3907; and Ma, D. R., et al. A review: the location, molecular characterisation and multipotency of hair follicle epidermal stem cells. Ann Acad Med Singapore. 2004; 33(6):784-788) and integrin 131 (ITGB1) (see, e.g., Bata-Csorgo, Z., et al. Kinetics and regulation of human keratinocyte stem cell growth in short-term primary ex vivo culture. Cooperative growth factors from psoriatic lesional T lymphocytes stimulate proliferation among psoriatic uninvolved, but not normal, stem keratinocytes. J Clin Invest. 1995; 95(1):317-327; Jones, P. H. Epithelial stem cells. Bioessays. 1997; 19(8):683-690; and Jones, P. H. et al. Stem cell patterning and fate in human epidermis. Cell. 1995; 80(1):83-93; and Jones, P. H. et al. Separation of human epidermal stem cells from transit amplifying cells on the basis of differences in integrin function and expression. Cell. 1993; 73(4):713-724) and, just prior to transplantation, the surface glycoprotein CD200 (see, e.g., Ohyama, M. Hair follicle bulge: a fascinating reservoir of epithelial stem cells. J Dermatol Sci. 2007; 46(2):81-89; and Ohyama, M. et al. Characterization and isolation of stem cell-enriched human hair follicle bulge cells. J Clin Invest. 2006; 116(1):249-260) together with keratin 18 (KRT18) (an epithelial marker) (Maurer, J. et al. Contrasting expression of keratins in mouse and human embryonic stem cells. PLoS One. 2008; 3(10):e3451) and the following hair follicle bulge stem cell (HFBSC) markers: transcription factor P63 (see, e.g., Ma, D. R. et al. (2004), and Pallegrini, G. et al. p63 identifies keratinocyte stem cells. Proc Natl Acad Sci USA. 2001; 98(6):3156-3161), keratin 15 (KRT15) ((see, e.g., Ma, D. R. et al. (2004), and Lyle, S., et al. The C8/144B monoclonal antibody recognizes cytokeratin 15 and defines the location of human hair follicle stem cells. J Cell Sci. 1998; 111(Pt 21):3179-3188), and keratin 19 (KRT19) (see, e.g., Ma, D. R. et al. (2004); Commo, S. et al. The human hair follicle contains two distinct K19 positive compartments in the outer root sheath: a unifying hypothesis for stem cell reservoir?Differentiation. 2000; 66(4-5):157-164; and Michel, M. et al. Germain L. Keratin 19 as a biochemical marker of skin stem cells in vivo and in vitro: keratin 19 expressing cells are differentially localized in function of anatomic sites, and their number varies with donor age and culture stage. J Cell Sci. 1996; 109(Pt 5):1017-1028) CD200, ITGA6, and ITGB1 on the surface of the hPSC likely interact with specific matrix receptors that not only mediate cell adhesion, survival, and proliferation (Rowland, T. J., Roles of integrins in human induced pluripotent stem cell growth on Matrigel and vitronectin. Stem Cells Dev. 2010; 19(8):1231-1240), but also entrance into a differentiation pathway that allows the emergence of HFBSCs in vitro and HFs in vivo. If the developmental stage and degree of responsiveness indicated by the presence of these markers is not achieved, HF replacement will not be possible. In other words, to yield optimal HFs in vivo, we make the developmental case for transplanting at Day 16 of differentiation—the point at which the hPSCs have lost almost all expression of pluripotency markers; have attained optimal expression levels of HFBSC markers but not yet progressed toward a keratinocyte fate; have not yet experienced downregulation of key integrins and surface CD200; and possess sufficient proliferative capacity to produce a well-populated graft. In fact, the necessity of these markers may be used to separate hiPSC derivatives that will yield HFs away from a heterogeneous population of derivatives that are disadvantageous for this therapeutic indication or perhaps even inimical (i.e., tumorigenic). The markers may also be used to identify trichogenic drugs.

Disclosed herein is a method of growing a hair follicle. The method comprises: (a) preparing human induced pluripotent stem cells (hiPSCs); (b) differentiating the hiPSCs into hair follicle bulge stem cells (HFBSCs); and (c) implanting the HFBSCs into skin of a subject.

In some embodiments, the hiPSCs have at least one, at least 2, at least 3, at least 4, at least 5, at least 6, at least 7, at least 8, or more markers selected from the group consisting of CD200, ITGA6, ITGB1, OCT4, NANOG, SOX2, TRA-1-60, TRA-1-81, and SSEA4. In some embodiments, the hiPSCs have 1, 2, 3, 4, 5, 6, 7, or 8 markers selected from the group consisting of CD200, ITGA6, ITGB1, OCT4, NANOG, SOX2, TRA-1-60, TRA-1-81, and SSEA4. In some embodiments, the hiPSCs have all of the markers of CD200, ITGA6, ITGB1, OCT4, NANOG, SOX2, TRA-1-60, TRA-1-81, and SSEA4.

In some embodiments, the HFBSCs have at least one, at least two, at least three, at least four, at least five, at least six, or more markers selected from the group consisting of CD200, ITGA6, ITGB1, KRT15, KRT18, KRT19, and P63. In some embodiments, the HFBSCs have one, two, three, four, five, or six markers selected from the group consisting of CD200, ITGA6, ITGB1, KRT15, KRT18, KRT19, and P63. In some embodiments, the HFBSCs have all markers of CD200, ITGA6, ITGB1, KRT15, KRT18, KRT19, and P63.

In some embodiments, the preparing in (a) comprises introducing, by electroporation, non-integrating episomal plasmid vectors encoding OCT3/4, SOX2, KLF4, L-MYC, LIN28 and an shRNA for human p531. In some embodiments, the electroporation is via a Neon transfection system.

In some embodiments, the differentiating in (b) comprises formation of embryoid bodies (EBs) in a floating culture. In some embodiments, the differentiating in (b) comprises plating the EBs onto coated plates. In some embodiments, the coated plates are collagen I coated plates.

In some embodiments, the method further comprises, prior to (c), (b1) differentiating the hiPSCs into keratinocytes. In some embodiments, the differentiating in (b1) comprises employing all-trans retinoic acid (ATRA) and L-ascorbic acid (L-AA) to induce the hiPSC to form ectoderm and then the addition of bone morphogenic protein-4 (BMP-4) and epidermal growth factor (EGF). In some embodiments, the differentiating in (b1) is according to a sequential differentiation protocol.

In some embodiments, the implanting in (c) comprises intradermal injection. In some embodiments, the implanting in (c) occurs at 15-19 days in vitro (DIV). In some embodiments, the implanting in (c) occurs at 16-18 DIV. In some embodiments, the implanting in (c) occurs at 15 DIV. In some embodiments, the implanting in (c) occurs at 16 DIV. In some embodiments, the implanting in (c) occurs at 17 DIV. In some embodiments, the implanting in (c) occurs at 18 DIV. In some embodiments, the implanting in (c) occurs at 19 DIV.

In some embodiments, the HFBSCs have not yet started expressing the keratinocyte associated molecules KRT5 and KRT14.

In some embodiments, the method further comprises treating hair loss and/or a condition in a subject in need thereof. In some embodiments, the condition is alopecia, ectodermal dysplasia, monilethrix, Netherton syndrome, Menkes disease, or hereditary epidermolysis bullosa.

In some embodiments, the subject is a human subject.

In one aspect, the present disclosure provides a composition, comprising (a) hair follicle bulge stem cells (HFBSCs); and (b) media. In some embodiments, the HFBSCs express markers CD200, ITGA6, ITGB1, KRT15, KRT18, KRT19, and P63.

In some embodiments, the HFBSCs do not express the keratinocyte associated molecules KRT5 or KRT14. In some embodiments, the HFBSCs is made by any method disclosed herein in this application.

In some embodiments, the composition is made by a process comprising: (i) preparing human induced pluripotent stem cells (hiPSCs); and (ii) differentiating the hiPSCs into the HFBSCs.

In some embodiments, the hiPSCs have one, two, three, four, five, six, or more markers selected from the group consisting of CD200, ITGA6, ITGB1, OCT4, NANOG, SOX2, TRA-1-60, TRA-1-81 and SSEA4. In some embodiments, the preparing hiPSCs in (i) comprises introducing, by electroporation, non-integrating episomal plasmid vectors encoding OCT3/4, SOX2, KLF4, L-MYC, LIN28 and an shRNA for human p531.

In some embodiments, the differentiating in (ii) comprises formation of embryoid bodies (EBs) in a floating culture. In some embodiments, the differentiating in (ii) comprises plating the EBs onto coated plates. In some embodiments, the coated plates are collagen I coated plates.

In another aspect, the present disclosure provides a method for hair follicle replacement, the method comprising: (a) obtaining human pluripotent stem cells (hPSCs); (b) differentiating the hPSCs, thereby producing differentiated hPSCs toward becoming keratinocytes; (c) capturing and isolating at least a portion of the differentiated hPSCs, wherein the portion of the differentiated hPSCs expresses hair follicle bulge stem cell markers (HFBSCM); and (d) transplanting the portion of the differentiated hPSCs into a patient in need thereof.

In some embodiments, the hPSCs are human induced pluripotent stem cells (hiPSCs).

In some embodiments, the hPSCs are hiPSC-derived hair follicle bulge stem cells (hiPSC-HFBSC). In some embodiments, the hiPSCs are derived from cells of the patient. In some embodiments, the hiPSCs are derived from cells of another subject.

In some embodiments, the cells of the patient are selected from the group consisting of fibroblasts, renal epithelial cells, and blood cells.

In some embodiments, the transplanting in (d) occurs at least 10, at least 11, at least 12, at least 13, at least 14, at least 15, at least 16, at least 17, at least 18, at least 19, at least 20, or more days after the differentiating in (b).

In some embodiments, the portion of the differentiated hPSCs is at a stage before the portion of the differentiated hPSCs has experienced downregulation of key integrins and key surface glycoproteins and before the portion of the differentiated hPSCs has started expressing keratinocyte-associated molecules.

In some embodiments, the key integrins comprise at least integrin α6 and/or integrin β1.

In some embodiments, the key surface glycoproteins comprise at least CD200.

In some embodiments, the stage is before the portion of the differentiated hPSCs has started expressing keratinocyte-associated molecules KRT5 and KRT14.

In some embodiments, the stage is after the portion of the differentiated hPSCs has expressed at least one of KRT15, KRT18, and KRT19, and P63.

In some embodiments, the transplanting in (d) comprises transplanting the portion of the differentiated hPSCs intradermally above the muscle coat.

In some embodiments, the capturing and isolating in (c) comprises flow cytometry.

In some embodiments, the capturing and isolating in (c) comprises capturing and isolating cells that co-express CD200 and integrin α6.

In some embodiments, the capturing and isolating in (c) comprises capturing and isolating cells that co-express CD200 and integrin β1.

In some embodiments, the patient in need thereof has alopecia or has lost hair follicles from an injury or burn.

Abbreviations used:

ATRA, all-trans retinoic acid; BMP, bone morphogenic protein; DAPI, 4, 6-Diamidino-2-phenylindole; DIV, days in vitro; EB, embryoid body; EGF, epidermal growth factor; FAD, a mixture of Ham's F12 and Dulbecco's Modified Eagle's Medium; FC, flow cytometry; hESCs, Human embryonic stem cells; HF, hair follicle; HFBSC, hair follicle bulge stem cell; hiPSC, human induced pluripotent stem cells; hiPSC-HFBSC, hiPSC-derived hair follicle bulge stem cells; hPSCs, human pluripotent stem cells; HSCA, human specific cytoplasmic antigen; HSNA, human specific nuclear antigen; ICC, immunocytochemistry; ITGA6, integrin a6; ITGB1, integrin 131; KRT, keratin; L-AA, L-ascorbic acid; LEF1, lymphoid enhancer-binding factor 1; MDC, mouse dermal cells; MSX2, msh homeobox 2; PHLDA1, Pleckstrin Homology Like Domain Family A Member 1; TDAG51, T-Cell Death-Associated Gene 51; TRPS1, trichorhinophalangeal syndrome type I.

Experimental Section

Material & Methods

Human Pluripotent Stem Cells (hPSCs)

Two types of hPSCs were used. For hESCs, the “gold standard” for hPSCs, the H9 line (Wicell) was employed. The hiPSCs were generated from normal human skin fibroblasts (obtained from de-identified donors) by using a Neon transfection system to introduce, by electroporation, non-integrating episomal plasmid vectors encoding OCT3/4, SOX2, KLF4, L-MYC, LIN28 and an shRNA for human p53 (see, e.g., Okita K., et al. A more efficient method to generate integration-free human iPS cells. Nat Methods. 2011; 8(5):409-412).

Characterization of hiPSCs

As previously described (see, e.g., Imaizumi Y. et al. Mitochondrial dysfunction associated with increased oxidative stress and alpha-synuclein accumulation in PARK2 iPSC-derived neurons and postmortem brain tissue. Mol Brain. 2012; 5:35; Ohta, S. et al. Generation of human melanocytes from induced pluripotent stem cells. PLoS One. 2011; 6(1):e16182; and Takahashi K., et al. Induction of pluripotent stem cells from fibroblast cultures. Nat Protoc. 2007; 2(12):3081-3089), hiPSCs colonies were characterized in vitro by both immune-cytochemistry (ICC) and flow cytometry (FC) for the presence of the following standard pluripotency markers using monoclonal antibodies against OCT4, NANOG, SOX2, TRA-1-60, TRA-1-81 & SSEA4. Pluripotency of the hiPSCs was further confirmed by proving their ability to form teratomas when injected into the flanks of immuno-incompetent (NSG) mice (Jackson Laboratory). The teratomas were examined histologically for the presence of cells representative of the 3 fundamental primitive germ layers (ectoderm, mesoderm, and endoderm), hence confirming pluripotency as disclosed herein.

Differentiation of hiPSCs into HFBSCs

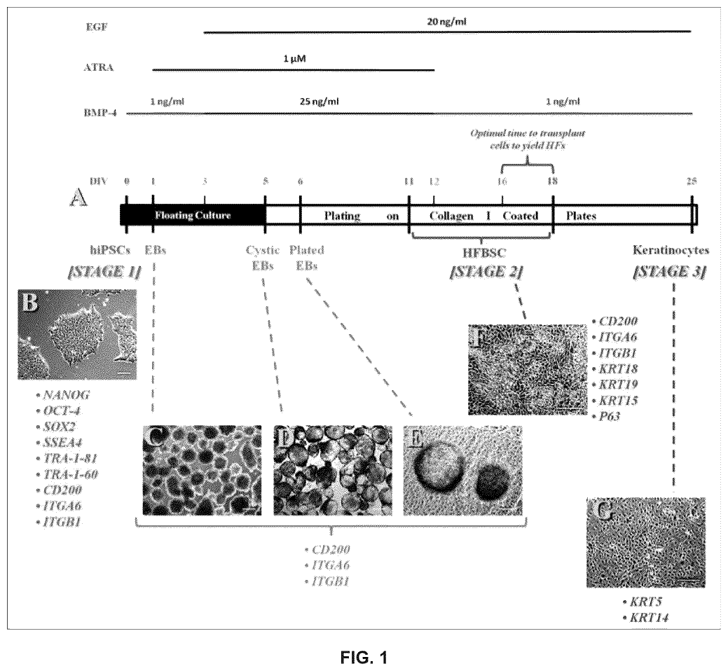

As a continuation of previous work (Ibrahim M. R, et al. Deriving keratinocyte progenitor cells and keratinocytes from human-induced pluripotent stem cells. Curr Protoc Stem Cell Biol. 2020; 54(1):e119), the overall strategy was to differentiate hiPSCs toward becoming keratinocytes but, just prior to that end-point, capture and isolate an intermediate transient progenitor cell population in vitro that expressed “HFBSCs” markers and could generated HFs. As schematized in FIG. 1 and detailed under hereinafter, the protocol was divided into 2 parts: first, the formation of embryoid bodies (EBs) in a floating culture followed, second, by plating the EBs onto collagen I coated plates. The protocol was repeated with at least 10 technical replications on at least 2 different hiPSC clones. To differentiate hiPSCs into keratinocytes, a sequential differentiation protocol is used to employ all-trans retinoic acid (ATRA) and L-ascorbic acid (L-AA) to induce hiPSC to form ectoderm, which were then differentiated into HFBSCs via the addition of bone morphogenic protein-4 (BMP-4) and epidermal growth factor (EGF). In this protocol, ectoderm is precluded from continuing to become neuroectoderm via BMP-4 suppression as disclosed hereinafter.

More detailed explanation of the protocol is shown in FIG. 1, the protocol is developed to generate HFBSCs and keratinocytes from hiPSCs. As shown in panel A, the protocol is divided into 2 parts: first, formation of floating EBs; second, plating of the EBs on collagen type I-coated plates. Throughout the differentiation protocol, the differentiated cells have 3-stage profile: Stage #1 is starting with the undifferentiated hPSCs, either hESCs or, as illustrated here, hiPSCs; Stage #2, the intermediate transient progenitor stage comprised of HFBSCs. These cells are the ones to be harvested and transplanted to yield HFs in vivo. If they are not harvested, they will continue to differentiate into keratinocytes (Stage #3) which have lost the competence to yield HF. To differentiate hiPSCs into keratinocytes, this sequential differentiation protocol that employs ATRA and L-AA to induce hiPSC was used to form ectoderm, which were then differentiated into HFBSCs via the addition of BMP-4 and EGF. In this schematic, the timeline for the appearance of each stage is shown, along with representative photomicrographs of cells at each stage (panels B-G), illustrating the morphological changes undergone by the hiPSCs and their derivatives over the course of the protocol, as well as the changes in marker expression that characterize each stage (juxtaposed to the respective photomicrograph). Panel B shows hiPSC colonies on DIV 0. Panel C shows that EBs are prepared manually on DIV 1. Panel D shows that nearly all EBs acquire a cystic morphology by DIV 5. Panel E shows that one day after plating of the EBs on DIV 6, the cells start to migrate out from the EB. Panel F shows that by DIV 11, the hiPSC-derived HFBSCs start to appear and persist until DIV 18. Panel G shows hiPSC-derived keratinocytes form by DIV 25. To obtain engraftable HFBSCs that will yield HFs in vivo following transplantation, the cells should be harvested, as indicated, on DIV 16-18. (Scale bar in Panel A is 30 rim. Scale bar in panels B-G is 100 rim).

In Vitro Characterization of hiPSC-Derived HFBSCs (“hiPSC-HFBSCs”)

ICC was performed using primary monoclonal antibodies against KRT18 (an epithelial marker); ITGA6, ITGB1, P63, KRT15, and KRT19 (HFBSCs markers); and KRT14 (a terminally differentiated keratinocyte marker). The temporal expression of CD200, ITGA6, and ITGB1 were monitored in differentiating hiPSCs using FC analysis. Relative gene expression of OCT4, P63, KRT15, KRT19, KRT8, KRT18, KRT5, and KRT14 in hiPSC-derived cells at days in vitro (DIV) 0, 11, 18, and 25 was assessed using qPCR analysis as described herein.

Co-Culture of hiPSC-HFBSCs & Mouse Dermal Cells (MDCs) in Vitro

hiPSC-HFBSCs were co-cultured with freshly isolated MDCs using 2 systems: (1) a transwell system, which allows 2 types of cells in monolayer to communicate with each other, but only via diffusible factors because they are separated by a porous membrane that allows the transit of only molecules but not cells; and (2) a 3-dimensional (3D) aggregate of the 2 cell types which allows cell-cell contact. Co-culturing was performed for 1 week, from DIV 11 to DIV 18. For the transwell co-cultures, 2.5×10⁵ hiPSC-HFBSCs were seeded onto a collagen coated surface in the bottom well while an equal number of MDCs were seeded onto permeable transwell inserts in the top well (Corning, Corning, NY). For the 3D co-culture system, equal numbers of hiPSC-HFBSCs and MDCs (2.5×10⁵) were mixed together in a Matrigel supported aggregate (1:1 mixture of Matrigel and “modified FAD medium”) supplemented with BMP-4 (25 ng/mL), ATRA (1 mM), and EGF (20 ng/mL). qPCR analysis of hair differentiation markers was performed on the co-cultured hiPSC-HFBSCs at DIV 18. The HF-associated genes assayed included KRT75, msh homeobox 2 (MSX2), lymphoid enhancer-binding factor 1 (LEF1), and trichorhinophalangeal syndrome type I (TRPS1) (see, e.g., Fantauzzo, K. A. et al. Dynamic expression of the zinc-finger transcription factor Trps1 during hair follicle morphogenesis and cycling. Gene Expr Patterns. 2008; 8(2):51-57; Gu, L. H. and Coulombe, P. A. Keratin expression provides novel insight into the morphogenesis and function of the companion layer in hair follicles. J Invest Dermatol. 2007; 127(5):1061-; 1073; Kobielak, A. and Fuchs, E. Links between alpha-catenin, NF-kappaB, and squamous cell carcinoma in skin. Proc Natl Acad Sci USA. 2006; 103(7):2322-2327; Kobielak, A. and Fuchs, F. The new keratin nomenclature. J Invest Dermatol. 2006; 126(11):2366-2368; and Rendl, M. et al. Molecular dissection of mesenchymal-epithelial interactions in the hair follicle. PLoS Biol. 2005; 3(11):e331).

Transplantation and Characterization of hiPSC-HFBSCs In Vivo

Patch grafting assays were performed as described previously (see, e.g., Kobayashi, T. et al. Canine follicle stem cell candidates reside in the bulge and share characteristic features with human bulge cells. J Invest Dermatol. 2010; 130(8):1988-1995; and Zheng, Y. et al. Organogenesis from dissociated cells: generation of mature cycling hair follicles from skin-derived cells. J Invest Derma-tol. 2005; 124(5):867-876). hiPSC-HFBSCs were combined on DIV 16 with freshly isolated MDCs from the backs of black C57BL/6 mice. Equal numbers of HFBSCs and MDCs (2.5×10⁶ each) were combined in 100 mL phosphate buffered saline (PBS) and injected intradermally into anesthetized (general) SCID mice above the muscle coat, where the cells could remain tightly packed and in contact with each other with little dispersion. Intradermal injection, in contrast to subcutaneous injection, not only prevented dispersion of the cells (cell aggregation and cell-cell interaction proving critical for organogenesis) but also provided a more appropriate microenvironment for the transplanted cells, the dermis being the natural milieu for growing hair.

Three-to-six weeks after implantation, the resulting growths were dissected, and processed for histological and immunohistochemical evaluation as described herein. The protocol was approved by the Sanford Burnham Prebys Institutional Animal Care and Use Committee.

Statistical Analysis

One-Way ANOVA was used to calculate the P values. A P-value of <0.05 was considered significant.

Reprogramming of Normal Human Skin Fibroblasts into Normal hiPSCs Using Episomal DNA Cocktail

To generate hiPSCs, normal human skin fibroblasts obtained from de-identified donors were electroporated using a Neon transfection system to introduce non-integrating episomal plasmid vectors encoding OCT3/4, SOX2, KLF4, L-MYC, LIN28 and an shRNA for human p53 (Okita et al. (2011))

A. Thawing & Maintaining Human Skin Fibroblast Cells
1. Prepare one 10 cm dish for each fibroblast cell line

B. Electroporation of Human Fibroblasts Using Neon Transfection System
1. Add 2 mL antibiotic free FB culture media to each well of a 6-well plate, prepare one well for each sample
2. Wash the cells 2× using DPBS
3. Add 2 mL 0.25% Trypsin to each dish
4. Incubate at 37° C. for 5 minutes
5. Add 5 mL fibroblast media to the dish
6. Collect the cell suspension to a 15 mL tube
7. Count cell number
8. Transfer 0.7 million cells to another 15 mL tube
9. Centrifuge at 200 g (1,000 rpm) for 5 minutes at room temperature (RT)
10. Remove the supernatant
11. Resuspend the cells using 2 mL DPBS
12. Centrifuge at 200 g (1,000 rpm) for 5 minutes at RT
13. During Centrifuge, mix cocktail DNA with Buffer R (included in Neon kit) according to the number of samples (see Table 1)

TABLE 1
Amount of cocktail DNA and Buffer R
according to the number of samples

| | For one | For five | For ten |
|---|---|---|---|
| | sample (μL) | samples (μL) | samples (μL) |
| Cocktail DNA | 4.4 | 22 | 44 |
| Buffer R | 105.6 | 528 | 1056 |

14. Aspirate the supernatant from the tube after centrifuge
15. Resuspend the cells with 110 μl DNA-buffer R mixture
16. Transfer the cells to an Eppendorf tube
17. Mix well with p200 pipet
18. Add 3 mL Buffer E2 to one neon cuvette
19. Put the neon cuvette into the neon pipette station
20. Using Neon pipette connected with an 100 ul Neon tip, aspirate the cocktail DNA/cell mixture
21. Put the Neon pipette into Neon cuvette placed in the Neon pipette station
22. Find the “Fibroblast Reprogramming” program saved in Neon transfection device: which is: 1650V, 10 ms, 3 time pulses
23. Push the “Start” button on the device
24. After the program completed, take the Neon pipette out from the device
25. Transfer the content instantly to the well with FB media without antibiotics
26. Change cuvettes and tips every time when transfect different cells
27. Rotate the plates many times then incubate at 37° C./CO₂ incubator

C. Amplification of the Transfected Cells
1. Change the media the next day using media with antibiotics
2. Keep the cells in fibroblast culture media for 7 days, media should be changed every 3-4 days

D. Re-Seeding the Transfected Cells on Feeders
1. On day 6, seed feeders to a 6-well plate
2. On day 7, wash the cocktail DNA transfected cells 2× with DPBS
3. Add 0.5 mL 0.25% trypsin solution to each well
4. Incubate at 37° C. for 5 minutes
5. Add 2 mL FB media to the well
6. Collect the cell suspension to a 15 mL tube
7. Count the cell number
8. Centrifuge at 200 g (1,000 rpm) for 5 minutes at RT
9. Remove the supernatant
10. Resuspend the cells using 1 mL FB media
11. Mix well with p1000 pipet
12. Adjust the volume, the concentration of the cells should be 1×10⁴/mL
13. Seed the transfected cells on feeders in FB media with antibiotics, at three densities: 0.5, 1, 2×10⁴ cells/well (6-well plate) are recommended
14. Rotate the plates many times then incubate at 37° C./CO₂ incubator

E. Reprogramming
1. On the next day, wash the cells once using DPBS
2. Change media to KOSR media
3. Change media every other day, for up to 6 weeks
4. Check iPSC colonies under stereo microscope, starting from week 3

F. Colony Pickup
1. Under a dissection microscope, manually pick up the colonies and transfer the colony to an Eppendorf tube,
2. Pipet up and down many times to break the colony into small clumps,
3. Prepare a MEF pre-seeded plate or a Matrigel coated plate,
4. Change media to KOSR medium supplemented ROCK inhibitor,
5. Transfer the colony from the eppendorf tube to the well,
6. Incubate overnight at 37° C. CO₂ incubator,
7. Change media the next day, with media without ROCK inhibitor.

Differentiation of hiPSCs into HFBSCs and Keratinocytes:

The goal of these procedures was to differentiate hiPSCs into keratinocytes and to capture the intermediate transient multipotent stem cell population in vitro called the “HFBSC”. To differentiate hiPSCs into keratinocytes, we used a sequential differentiation protocol that employs ATRA and L-AA to induce hiPSC to form ectoderm, which was then differentiated into KPCs and keratinocytes. In this protocol ectoderm is precluded from continuing to become neuroectoderm via BMP4 suppression. The protocol is divided into 2 parts; formation of EBs in floating culture and plating of the obtained EBs on collagen I coated plates.

A. Reagents
Embryoid Body (EB) formation medium (see “Media” section below for composition)
Modified FAD medium (see “Media” section below for composition)
Phosphate-buffered saline (PBS)
Rock inhibitor (Y-27632) (STEMCELL technologies, Cat #72302)
Collagen I (Corning, Cat #354236)
Bone morphogenetic protein-4 (BMP-4) (R&D systems, Cat #314-BP)
All-trans retinoic acid (ATRA) (1 μM) (Sigma-Aldrich, Cat #R2625)
Epidermal growth factor (EGF) (20 ng/mL) (Sigma-Aldrich, Cat #E9644)
Dulbecco's Modified Eagle Medium (DMEM) (Life Technologies, Cat #11965)
Ham's F12 Nutrient Mix, GlutaMAX™ (Life Technologies, Cat #31765)
Fetal Bovine Serum (FBS) (Thermo Fisher Scientific, Cat #MT35010CV)
Insulin (Sigma-Aldrich, Cat #19278)
Hydrocortisone (Santa Cruz, Cat #sc-300810)
Cholera toxin (Sigma-Aldrich, Cat #C8052)
Triiodothyronine (T3) (Sigma-Aldrich, Cat #T6397)
L-ascorbic acid (L-AA)(Sigma-Aldrich, Cat #255564)
Adenine (Sigma-Aldrich, Cat #2786)
Defined Keratinocyte Serum Free Medium (DKSM) (Thermo Fisher Scientific, Cat #10744019)
Keratinocyte Serum Free Medium (KSM) (Thermo Fisher Scientific, Cat #17005042)

“DKSM medium” is a commercially-available serum-free medium optimized for the isolation and expansion of human keratinocytes without the need for bovine pituitary extract (BPE) supplementation or the use of fibroblast feeder layers. The abbreviation DKSM stands for “Defined Keratinocyte Serum Free Medium” and was obtained from Thermo Fisher Scientific (Cat #10744019). In this formulation, the growth-promoting activity of BPE has been emulated instead by defined trophic factors that can maintain cell morphology, physiological markers, and growth of the cultured keratinocytes. DKSM has a low calcium concentration (<0.1 mM) which maintains keratinocytes in an undifferentiated proliferative stage.

B. Media
(i) Fibroblast (FB) Culture Medium (per 1,000 mL):
   900 mL DMEM
   100 mL FBS
   10 mL Non-essential amino acids (NEAA) (Life Technologies, Cat #11140-050)
   10 mL Anti-Anti (Antibiotic-Antimycotic) (Life Technologies, Cat #: 15240062)
Filter, sterilize, and store at 4° C.; media will last for up to 14 days.
(ii) Another FB culture medium (per 1,000 mL):
   900 mL DMEM
   100 mL FBS
   10 mL NEAA (Life Technologies, Cat #11140-050)
Filter, sterilize, and store at 4° C.; media will last for up to 14 days.
(iii) Knockout serum replacement (KOSR) medium:
   400 mL DMEM/F12
   100 ml KOSR (Fisher Scientific, Cat #A3181502)
   5 mL NEAA (Life Technologies, Cat #11140-050)
   0.9 mL Beta-ME
   5 mL Anti-Anti (Antibiotic-Antimycotic) (Life Technologies, Cat #: 15240062)
   50 μlb FGF (100 ng/μL)
Can store up to 4 weeks at 4° C. or up to 4 months at −20° C.
(iv) EB formation medium:
   400 mL DMEM/F12
   100 mL KOSR
   5 mL NEAA
   5 mL Anti-Anti (Antibiotic-Antimycotic)
   0.9 mL Beta-ME
   0.5 mL Rock inhibitor (Y-27632)
   BMP-4 (1 ng/mL)
   No FGF2
Filter through a 0.22 μm filter
(v) EAD medium:
   3:1 mixture of DMEM and Ham's F12 medium
(vi) Modified FAD medium:
   3:1 mixture of DMEM and Ham's F12 medium (standard FAD medium)
   FBS (2%)
   Insulin (5 μg/mL)
   Hydrocortisone (0.5 μg/mL)
   Cholera toxin (10⁻¹⁰ mol/L)
   Triiodothyronine (1.37 ng/mL)
   L-AA (0.3 mmol/L)
   Adenine (24 μg/mL)

Day 0:

Embryoid Body (EB) Formation
Passage 20-35 healthy hiPSC lines were selected.
On day 0, when hiPSC colonies had reached 80 to 90% confluence, wash with DBPS.
Separate hiPSC colonies manually into small clusters.
Use P-1000 to pipet up and down several times to reduce the cluster size (about 50-100 cells/cluster).
The clusters were cultured in ultra-low adherence 6-well plate in EB suspension culture medium supplemented with ROCK inhibitor “Y-27632” (1 W/ml), BMP-4 (1 ng/ml) without FGF2, in a volume of 3 mL/well.
ROCK inhibitor: Improves EB formation

On the 2^{nd} day (Day 1):
Check EBs under stereo microscope.
Collect EBs in a 15 mL conical tube.
Wait 10 minutes to allow EBs to settle down by gravity.
Remove supernatant.
Resuspend in modified FAD medium supplemented with BMP-4 (1 ng/ml) and ATRA (1 μM).
After resuspension, plate the EBs in an ultra-low adherence 6-well plate using 10 ml sterile serological pipettes, aspirate quickly but gently (final volume 3 mL/well)
Note: Quickly but gently move the plate in side-to-side, forward-to back motions to evenly distribute the EBs within the wells and to avoid EBs sticking together.

On Day 2:
EBs were collected in a 15 mL conical tube and media were changed to modified FAD medium supplemented with BMP-4 (1 ng/mL) and ATRA (1 μM)

From Day 3 to Day 7:
Use modified FAD medium supplemented with BMP-4 (25 ng/mL), ATRA (1 μM) and EGF (20 ng/mL)
Note: Gentle shaking of the plate 1-2 times per day for the first few days of suspension culture may help to distribute the EBs in the well, and avoid EBs sticking together. Note: On day 5 (6th day) the floating EBs are plated on collagen I coated plates.
Note: Coating plates with collagen I: Dilute collagen I to 50 μg/mL with 0.02 N acetic acid. Add sufficient diluted collagen I to coat dishes, 5-10 μg/cm surface. Incubate the coated plates for 2-3 hours at RT. Aspirate remaining solution and rinse wells with PBS 2 times remove acid. Plates can be used instantly or stored at 2-8° C. for up to one week under sterile conditions

From Day 8 to Day 11:
Day 8: FAD medium: Defined Keratinocyte Serum Free Medium (DKSM)=3:1
Day 9: FAD medium: DKSM=1:1
Day 10: FAD medium: DKSM=1:3
Day 11: DKSM only
From day 8 to day 11 the used medium was supplemented with insulin (5 μg/mL), hydrocortisone (0.5 μg/mL), cholera toxin (10⁻¹⁰ mol/l), triiodothyronine (1.37 ng/mL), L-AA (0.3 mmol/l), and adenine (24 μg/mL).
Small molecules and growth factors, BMP-4 (25 ng/mL), ATRA (1 μM) and EGF (20 ng/mL) were added to the medium.

From Day 12 to Day 25
Used equal amounts of DKSM and Keratinocyte Serum Free Medium (KSM) (DKSM: KSM 1:1) supplemented with BMP-4 (1 ng/mL) and EGF (20 ng/mL)
The protocol was repeated with at least 10 technical replications on at least 2 different hiPSC clones, hence biological replicates

To passage hiPSC-HFBSCs:
1. Remove the medium.
2. Wash with PBS
3. Add 0.5 ml of pre-warmed 0.05% trypsin
4. Incubate hiPSC-KPC for 5 minutes at 37° C. with 5% CO₂ and 95% humidity.
5. Remove the culture plate from the incubator and place in the hood.
6. Dislodge the cells by hitting the plate several times against the heal of your hand 3-5 times.
7. Transfer the cell contents into the 15 mL tube with 5 ml pre-warmed media.
8. Centrifuge at 200 g (1,000 rpm) for 5 minutes at RT
9. Remove the supernatant.
10. Resuspend the cells and plate on fresh collagen I coated plates.

Immunocytochemistry (ICC)

Materials:
24-well tissue culture plates
PBS (without Mg2+ or Ca2+)
4% Paraformaldehyde in PBS (4% PFA)
Triton X-100
Bovine serum albumin (BSA)
DAPI
Mounting medium (optional)

Prepare the Blocking Buffer (BB):
Use PBS (the amount necessary to complete the ICC). Add 5-10% of BSA.
   For surface antigens, the BB is ready to use (RTU).
   For intracellular antigens, add 0.1% of Triton X-100.
Store BB up to 6 months at 4° C.

Procedure:
1. Passage and culture cells in a 24-well plate till ready for ICC analysis.
Note: Use matrigel coated plates for hiPSC.
Note: Use collagen I coated plates for hiPSC-derived KPC and keratinocytes.
2. Wash each well 3× using 0.5 mL of RT PBS.
3. Fixation: use 0.5 mL 4% PFA/well and keep at RT for 20 minutes.
4. Remove the PFA then wash each well 3× with 0.5 mL PBS for 5 minutes.
5. Add 0.5 mL/well of BB to block non-specific antibody binding and keep at RT for 60 minutes.
6. Prepare the primary antibody by diluting it in BB (see Table 2).

TABLE 2
List of antibodies used in immunocytochemistry (ICC)

| Antibody Name | Host | Dilution | Company | Cat # |
|---|---|---|---|---|
| OCT4 | Rabbit | 1:200 | Cell Signaling | 2840 |
| NANOG | Rabbit | 1:200 | Cell Signaling | 4903 |
| SOX2 | Rabbit | 1:200 | Cell Signaling | 3579 |
| TRA-1-60 | Mouse | 1:200 | Cell Signaling | 4746 |
| TRA-1-81 | Mouse | 1:200 | Cell Signaling | 4745 |
| SSEA4 | Mouse | 1:200 | Cell Signaling | 4755 |
| CD200 | Mouse | 5:200 | eBioscience | 12-9200-41 |
| ITGA6 | Rat | 1:200 | eBioscience | 11-0495-80 |
| ITGB1 | Mouse | 5:200 | eBioscience | 17-0299-41 |
| Keratin15 | Rabbit | 1:100 | Abcam | ab52816 |
| Keratin15 | Mouse | 1:200 | NeoMarkers | MS-1068-P0 |
| Keratin19 | Mouse | 4:200 | eBioscience | 14-9898-80 |
| Keratin18 | Rabbit | 1:200 | Abcam | ab133263 |
| P63 | Rabbit | 1:200 | STEMCELL | 60154 |
| Keratin14 | Rabbit | 1:200 | Abcam | ab51054 |

7. Remove the BB and add the diluted primary antibody (200 μL) to each well then keep the plate at 4° C. overnight.
8. Wash each well 3× using 0.5 mL PBS for 5 minutes.
9. Prepare the secondary antibody by diluting it in BB according to the manufacturer's instructions.
10. Add the diluted secondary antibody (200 μL) to each well and keep at RT for 60 minutes while protecting the plate from light by wrapping with aluminum foil.
11. Wash each well 3× using 0.5 mL PBS for 5 minutes (protect the plate from light).
12. Prepare diluted DAPI (0.2 mg/mL) in PBS (1:10,000), for nuclear visualization during fluorescent imaging, and add 0.5 mL per well for 10 min at RT (protect the plate from light by covering with aluminum foil).
13. Wash each well once using 0.5 mL PBS for 5 minutes (protect the plate from light).
14. Aspirate any PBS remaining in the wells (You can add mounting medium “1-2 drops/well”.
15. Examine the plate under the fluorescence microscope and take photos for each well.

Immunofluorescence Staining of Cells for Flow Cytometry (FC)

Materials:
Flow Buffer
Trypan blue
10% formalin solution
PBS
15 mL conical tubes
12×75 mm round-bottom tubes

Prepare the Flow Buffer:
99 mL PBS (1×)
1 mL FBS
0.1 mL Sodium Azide (100%)
   For extracellular antigens, the flow buffer is RTU.
   For intracellular antigens, add Saponin to a final concentration of 0.1%.
Store flow buffer at 4° C.

Procedure:
1. Prepare a single cell suspension then collect the dissociated cells in a 15 mL conical tube.
2. Count cell number using trypan blue and a hemacytometer.
3. Centrifuge at 300×g for 5 minutes at 4° C.
4. Remove the medium then flick the tube to disrupt the cell pellet.
   For extracellular antigens, resuspend the cell pellet using 3 mL PBS.
   For intracellular antigens, resuspend the cell pellet in 2 mL 10% formalin solution and keep for 15 min at RT.
5. Centrifuge at 300×g for 5 minutes at 4° C.
6. Remove the medium then flick the tube to disrupt the cell pellet.
7. Using appropriate amount of the flow buffer, set the cell suspension to a concentration of 2×106 to 1×107 cells/mL. Keep the cells on ice.
8. For each sample, add 100 μL of the cell suspension to a 12×75 mm round-bottom tube.
9. Add the suitable amount of primary antibody to each sample (see Table 3).

TABLE 3
List of antibodies

| Antibody Name | Diluti | Company | Cat # |
|---|---|---|---|
| CD200 | 5:200 | eBioscience | 12-9200-41 |
| ITGA6 | 1:200 | eBioscience | 11-0495-80 |
| ITGB1 | 5:200 | eBioscience | 17-0299-41 |

10. Keep on ice for 1 hour while covering the tubes from light.
11. Add 4 mL flow buffer.
12. Centrifuge at 300×g for 5 minutes at 4° C.
13. Remove the supernatant then flick the tube to disrupt the cell pellet.
14. Add the proper volume of flow buffer to each tube.
15. Analyze the cells by FC within 4 hours.
Stained cells were analyzed using a LSRFortessaflow cytometer (BD Biosciences, San Jose, CA). Data were then analyzed using FACSDiVa v8.0.2 software (BD Biosciences, San Jose, CA).

Quantitative Reverse Transcription-Polymerase Chain Reaction (qRT-PCR)

RNA Isolation: (RNeasy Mini Kit, Qiagen, Cat. #74104)
1. Collect cells (max. of 1×10⁷) as a cell pellet then add 350 μL of Buffer RLT, vortex.
2. Add 1 volume of 70% ethanol to the lysate, then pipet to mix well. NO centrifugation.
3. Take 700 μL of the sample (include any precipitate), to a RNeasy Mini spin column placed in a 2 mL collection tube. Close the lid, and centrifuge for 15 s at ≥8000×g. Discard the flow-through.
4. Add 700 μL Buffer RW1 to the RNeasy spin column. Close the lid, and centrifuge for 15 s at ≥8000×g. Discard the flow-through.
5. Add 500 μL Buffer RPE to the RNeasy spin column. Close the lid, and centrifuge for 15 s at ≥8000×g. Discard the flow-through.
6. Add 500 μL Buffer RPE to the RNeasy spin column. Close the lid, and centrifuge for 2 min at ≥8000×g.
Optional: Place the RNeasy spin column in a new 2 mL collection tube. Centrifuge at maximum speed for 1 min to dry the membrane.
7. Place the RNeasy spin column in a new 1.5 mL collection tube. Add 30-50 μL RNase-free water directly to the spin column membrane. Close the lid, and centrifuge for 1 min at ≥8000×g to elute the RNA.
8. If the expected RNA yield is >30 μg, repeat step 7 using another 30-50 μL of RNase-free water, or using the eluate from step 7 (if high RNA concentration is required). Reuse the collection tube from step 7.

Synthesize first-strand cDNA (SuperScript™ III First-Strand Synthesis SuperMix for qRT-PCR, Invitrogen, Catalog #11752-050)
1. Combine the following kit components in a tube on ice (see Table 4). For multiple reactions, a master mix without RNA may be prepared:

TABLE 4
Reagents for 1^{st} strand cDNA

| Component | Amount |
|---|---|
| 2× RT Reaction Mix | 10 μL |
| RT Enzyme Mix | 2 μL |
| RNA (up to 1 μg) | x μL |
| DEPC-treated water | to 20 μL |

2. Gently mix tube contents then incubate at 25° C. for 10 minutes.
3. Incubate tube at 50° C. for 30 minutes.
4. Terminate the reaction at 85° C. at 5 minutes, then chill on ice.
5. Add 1 μL (2 U) of E. coli RNase H and incubate at 37° C. for 20 minutes.
6. Use diluted or undiluted cDNA in qPCR, or store at −20° C. until use.

Real-time PCR (SensiFAST™ SYBR® No-ROX Kit, Bioline, BIO-98005) Reaction mix composition: Prepare a PCR master mix. The volumes given in Table 5 are based on a standard 20 μL final reaction mix and can be scaled accordingly.

TABLE 5
Reagents for Real Time PCR

| | | |
|---|---|---|
| 2× SensiFAST SYBR ® No-ROX Mix | 10 μL | 1× |
| 10 μM forward primer | 0.8 μL | 400 nM |
| 10 μM reverse primer | 0.8 μL | 400 nM |
| Template | up to 8.4 μL | |
| H₂O | As required | |
| | 20 μL Final volume | |

The conditions for 3-step cycling are presented in Table 6.

TABLE 6
PCR Cycles

| Cycles | Temperature | | Time | | Notes |
|---|---|---|---|---|---|
| 1 | 95° | C. | 2 | min | Polymerase activation |
| 40 | 95° | C. | 5 | s | Denaturation |
| | 60-65° | C. | 10 | s | Annealing |
| | 72° | C. | 5-20 | s | Extension (acquire at end of step) |

Messenger RNA expression levels were normalized to 18S expression based on the ACT method and calculated based on 2-ACT.
The results are presented as mean±standard error of means (SEM). Statistical significance of the differences in RT-PCR analysis was determined using One-Way ANOVA test with P<0.05 considered significant.
The sequences of the primers are presented in Table 7.

TABLE 7
Sequence of the PCR primers

| Gene | Forward sequence | Reverse sequence |
|---|---|---|
| OCT4 | CCTCACTTCACTGCACTGTA | CAGGTTTTCTTTCCFTAGCT (SEQ |
| | (SEQ ID NO: 1) | ID NO: 9) |
| TP63 | CCGCCGTCCAATTTTAATCA | CGTCGGCCCAGGACTTG (SEQ ID |
| | (SEQ ID NO: 2) | NO: 10) |
| KRT15 | CTTCAGGAGGTGGTGGTAGCA | CACCTGTCCATCCACTGACTCTT |
| | (SEQ ID NO: 3) | (SEQ ID NO: 11) |
| KRT19 | AACCAAGTTTGAGACGGAACA | GAGCGGAATCCACCTCCAC (SEQ |
| | G (SEQ ID NO: 4) | ID NO: 12) |
| KRT8 | GATCGCCACCTACAGGAAGCT | ACTCATGTTCTGCATCCCAGACT |
| | (SEQ ID NO: 5) | (SEQ ID NO: 13) |
| KRT18 | GAGTATGAGGCCCTGCTGAAC | GCGGGTGGTGGTCTTTTGGAT |
| | AT CA (SEQ ID NO: 6) | (SEQ ID NO: 14) |
| KRT5 | ATCTCTGAGATGAACCGGAT | CAGATTGGCGCACTGTTTCTT |
| | GAT C (SEQ ID NO: 7) | (SEQ ID NO: 15) |
| KRT14 | GGCCTGCTGAGATCAAAGACT | CACTGTGGCTGTGAGAATCTTGT |
| | AC (SEQ ID NO: 8) | T (SEQ ID NO: 16) |

Patch Assay
Mice: C57BL/6
Site: Back
First Day:

Requirements:
1. 50 mL conical tubes (PBS with antibiotics)
2. Empty 50 mL conical tube
3. Surgical instruments
4. Surgical cover
5. Base for 50 mL conical tubes.

Procedure: Sterilize the Animal (Betadine and Ethanol 70%)
Back skins were isolated, washed in PBS with antibiotic/antimycotic (5 times for 5 minutes shaking),
Transfer placed on ice,
Incubate in 0.01% Dispase overnight at 4° C.

Second Day (Day of Injection):
Epidermal layers of skin were isolated with forceps, washed in PBS (with anti/anti) for 5 minutes and incubated in 0.1% Trypsin-EDTA solution at 37 C for 8 minutes.
Dermal layers: homogenized with scissors and digested with 0.1% Trypsin EDTA (or collagenase) solution at 37° C. for 45 minutes.
Add MEF media to block enzyme activity.
Pipette epidermis and dermis vigorously for 10 minutes.
Cell suspension was isolated with cell strainer.
Centrifuge.
Wash in PBS.
Centrifuge.
Resuspend in PBS.
In all experiments, approximately equal numbers of epithelial and dermal cells (2.5 million each) were combined in 100 ul BPBS and kept on ice until transplanted intradermal into SCID mouse under general anesthesia.

Immunohistochemistry (IHC)

Using Shandon Apparatus Technique

Materials Required:
Slides
DPBS
BSA
Goat Serum
Triton-X, antibodies
Coverslips
Mounting media (FluorSave)
Hoechst (DAPI).

Procedures:
1. Section tissue (using cryostat or microtome) and place tissue in middle &/or lower half of the glass slide.
2. Label slides with tissue info, primary antibodies and concentration used, date, name.
3. Place slides into plastic slide holders against holder feet with tissue facing plastic/middle. Holding slides and holders together, place the slides and holders in the staining box.
4. Rehydrate tissue and clear out excess paraffin/OCT with 200 μL DPBS quick wash.
5. Wash 2× for 5 minutes with 200 μL DPBS.
6. Prepare enough blocking/permeabilization solution: 3% BSA, 3% Goat Serum, 0.1% Triton X-100 in DPBS.
7. Add 150 μL of blocking/permeabilization solution to slides for 1 hour at RT.
8. Prepare primary antibody solutions in blocking solution (1:250)
Note: If staining for multiple proteins, make sure the primary antibodies are different species.
Note that the primary antibodies used are those in Table 2 except for the addition of the human-specific antibodies listed in Table 8 used following in vivo transplants to identify human-derived cells.

TABLE 8
Human-Specific Antibodies Used for Immunohistochemistry
Following Transplantation

| Antibody Name | Dilution | Company | Cat # |
|---|---|---|---|
| Anti-human Nuclei | 1:250 | Sigma-Aldrich | MAB-1281 |
| antibody (HuNu) | | (Merck) | |
| STEM121 | 1:500 | Takara Bio | Y40410 |

9. Wet and fold large kimwipes with deionized water at sink and place at top corners of Shandon rack foil box cover piece so that there is a good seal with the lid and box for humidity maintenance. Place box with slides and lid in cold room on a stationary bench/table overnight.
10. Next day, take slide box into lab and RT. Wash 3× for 5 minutes using 200 μL DPBS.
11. Prepare secondary antibody solution in same blocking/permeabilization solution (1:1000).
Note: If staining for multiple proteins, make sure the primary antibodies target correct species at different wavelengths/colors. Example: Goat anti Mouse 488 and Goat anti Rabbit 555
12. Add 150 uL of appropriate secondary antibody solution to slides and incubate for 60 minutes at RT (protect the plate from light by covering with aluminum foil).
13. Do 1× quick wash using 200 μL DPBS, then 3×5-minute 200 μL DPBS washes, prepare DAPI solution.
14. Hoechst/DAPI=1:5000 in DPBS. Add 150 μL to slides for 15 minutes at RT (protect the plate from light by covering with aluminum foil).
15. Do 1× wash using 200 μL DPBS for 5 minutes (protect the plate from light by covering with aluminum foil).
16. Very carefully remove slide from box and the plastic slide holder. Watch to make sure tissue stays flat and straight on slide.
17. Very carefully using small kimwipe to dab dry surrounding DPBS liquid around tissue, but since you cannot dry everything, place the tissue face up in a drawer to let dry and protect from light. Check after 15 minutes to see if dry enough to coverslip.
18. Add two small drops of FluorSave mounting media. Make sure there is enough mounting media to cover the tissue and make sure there are no bubbles.
19. Place coverslip using one edge as a fulcrum to slowly apply coverslip. Carefully tap out any bubbles that are made by adding the coverslip by using the end of a paintbrush or something similar.
20. Place the cover-slipped slides in a drawer to protect from light for one hour for mounting media to dry and solidify.
21. Can start to image with fluorescent or confocal microscope. It may help to clean the coverslip and slide exteriors with gently wiping with “Sparkle” solution or just try with 70% Ethanol spray.
22. Short term storage of slides in slide box in 4° C. fridge. Long term storage at −80° C. freezer.

Results

One aspect of the present disclosure is to exploit insights from the dynamic up- and down-regulation of the key developmental molecules that determine HF lineage commitment by hPSCs. The insights can ascertain the precise differentiation stage and molecular requirements, as indicated by surrogate biomarkers for successful HF replacement in vivo based on the conversion of hiPSCs into engraftable hair generating cells. The results can produce a strategy that entailed differentiating hiPSCs toward becoming keratinocytes in vitro but, just prior to that end-point, capturing and isolating an intermediate transient progenitor cell population, which expressed HFBSC-associated markers and could generate HFs in vivo before going on to become (which may be less desirable) non-HF-producing keratinocytes (See FIG. 1). Cells at earlier or later development stages may not yield HFs, suggesting that these intermediate stage cells were bona fide HFBSCs. These data are applicable to all hPSCs, including enabling the use of hiPSCs. hiPSCs generated from prospective transplant recipients according to the present disclosure may avoid immunologic incompatibility, thereby having clinical use and applications.

For more details, see Ibrahim, M. R., et al. (2021). The Developmental & Molecular Requirements for Ensuring that Human Pluripotent Stem Cell-Derived Hair Follicle Bulge Stem Cells Have Acquired Competence for Hair Follicle Generation Following Transplantation. Cell transplantation, 30, 9636897211014820, the content of which is entirely incorporated herein by reference.

Generation and Characterization of hiPSCs

hiPSCs were generated de novo by electroporating into de-identified normal human skin fibroblasts, non-integrating episomal plasmid vectors encoding OCT3/4, SOX2, KLF4, L-MYC, LIN28 and an shRNA for human p53 (Okita, K. (2011)). The hiPSC clones exhibited appropriate hPSC morphology and were isolated about 21-45 days post-transfection. Fourteen distinct clones were generated following 2 independent rounds of transfections. Of these 14 clones, 2 unrelated clones were selected at random for the studies described here.

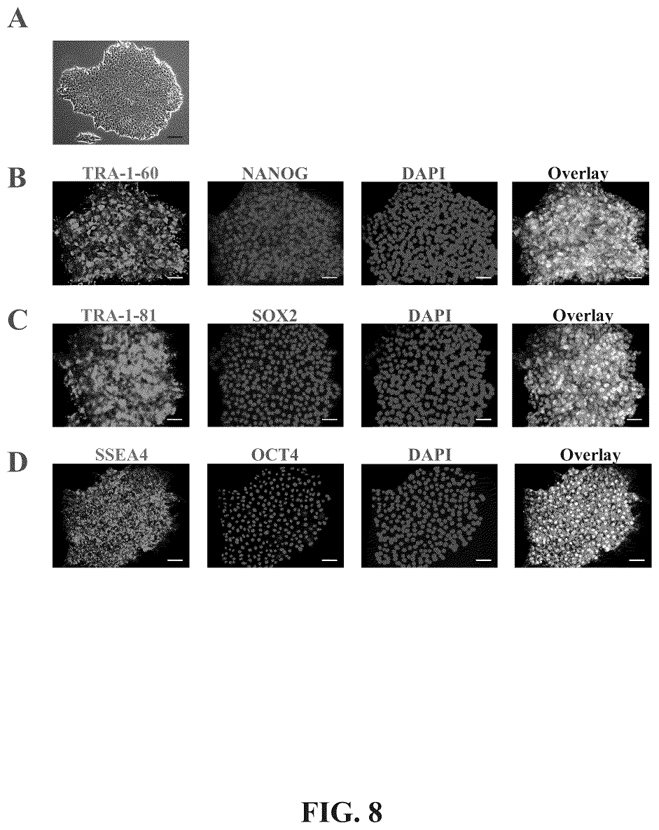

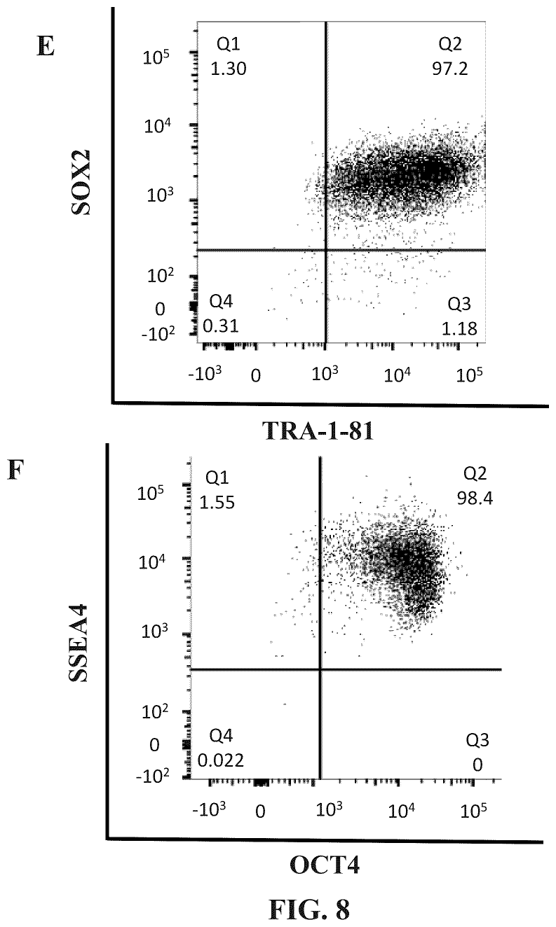

Given that hESCs are the gold standard for hPSCs, hESCs were also studied through all subsequent steps in parallel with the hiPSCs for validation. Like the hESCs, the new hiPSCs expressed the following panel of pluripotency immunomarkers as determined by ICC and FC: the nuclear transcription factors NANOG, SOX2, and OCT4; the surface markers TRA-1-60, TRA-1-81, and SSEA4. Pluripotency of the hiPSC clones was further confirmed by demonstrating their ability to form teratomas containing cells representative of all 3 fundamental primitive germ layers in immunocompromised NSG mice as shown in FIG. 8.

FIG. 8 shows characterization of hiPSCs derived from primary human fibroblasts. FIG. 8 Panel A shows morphology of hiPSC colonies emulates that of hESC colonies (Scale bar, 30 m). FIG. 8, Panels A-D show immunocytochemical analysis: hiPSC clones express markers definitive of pluripotent cells, TRA-1-60, TRA-1-81, SEAA4, and OCT4, as well as NANOG and SOX2, respectively. Secondary antibodies against surface markers (TRA-1-60, TRA-1-81 and SSEA4) are conjugated with FITC (green), while secondary antibodies against nuclear markers (NANOG, SOX2, and OCT4) are conjugated with PE (red). DAPI (blue) stains the nucleus of all cells in the field (Scale bar, 30 m). According to FIG. 8 Panel E and F, slow cytometry also shows that the hiPSC clones express markers indicative of the pluripotent state, including, TRA-1-81 & SOX2, and SSEA4 & OCT4. hESCs, human embryonic stem cells; hiPSCs, human induced pluripotent stem cells.

Generation, Characterization, & Differentiation of hiPSCs into HFBSCs

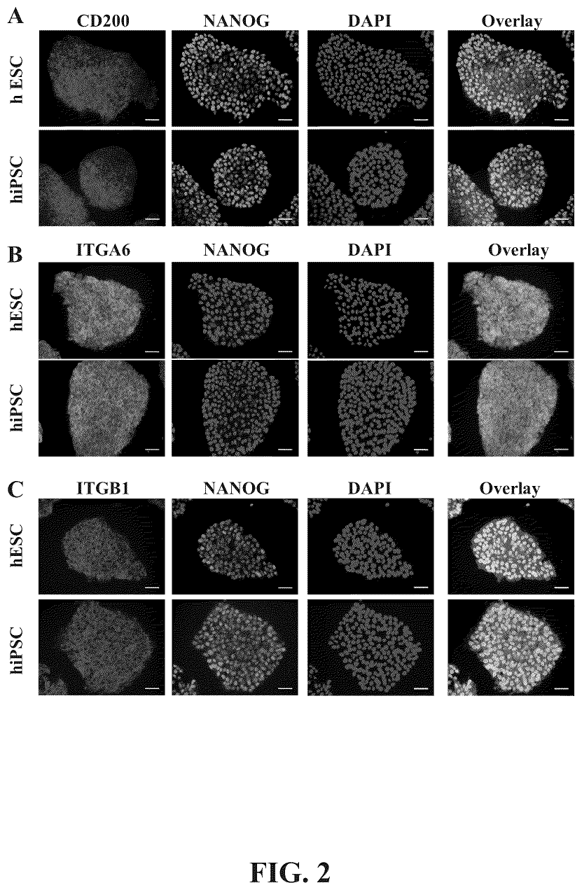

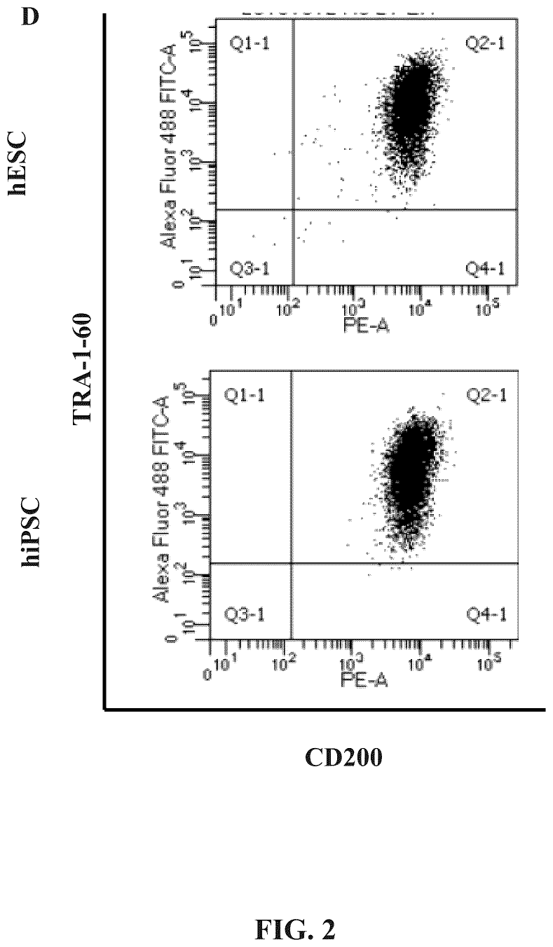

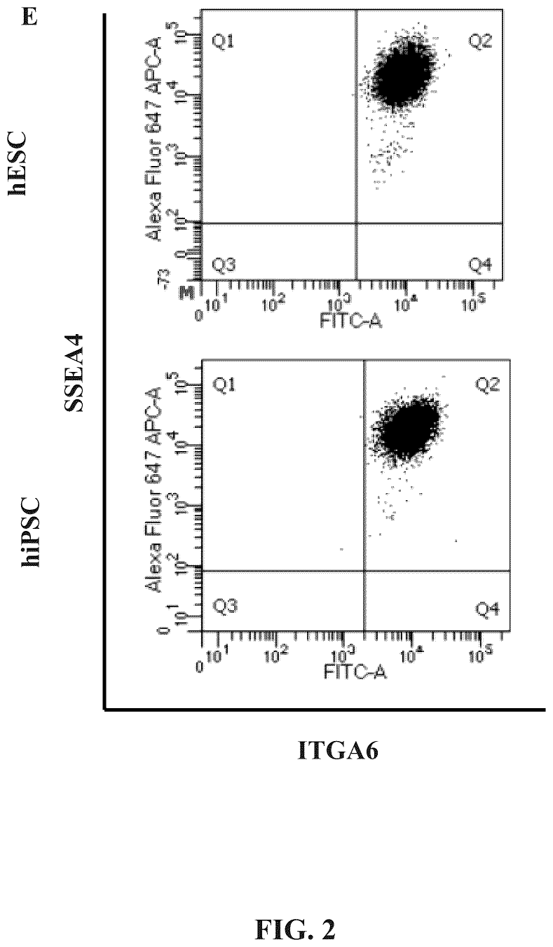

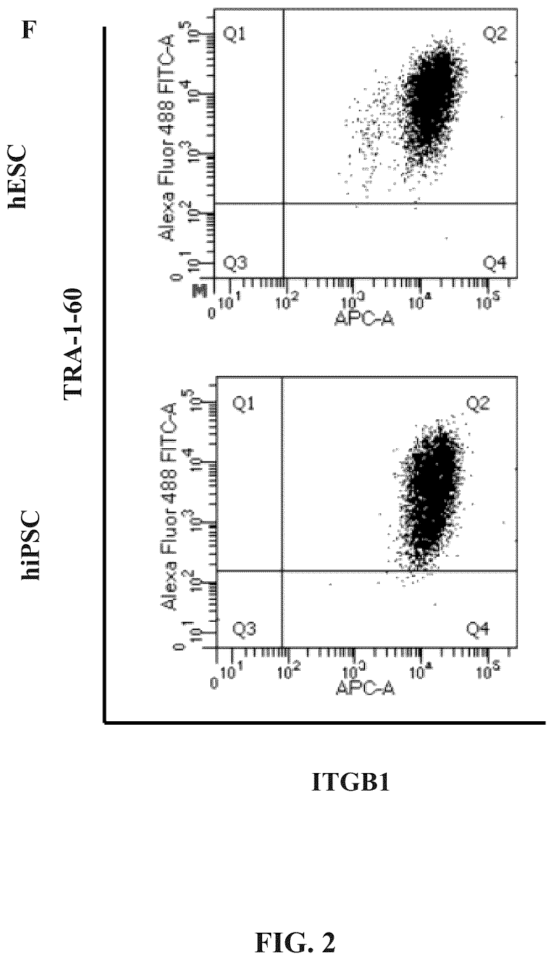

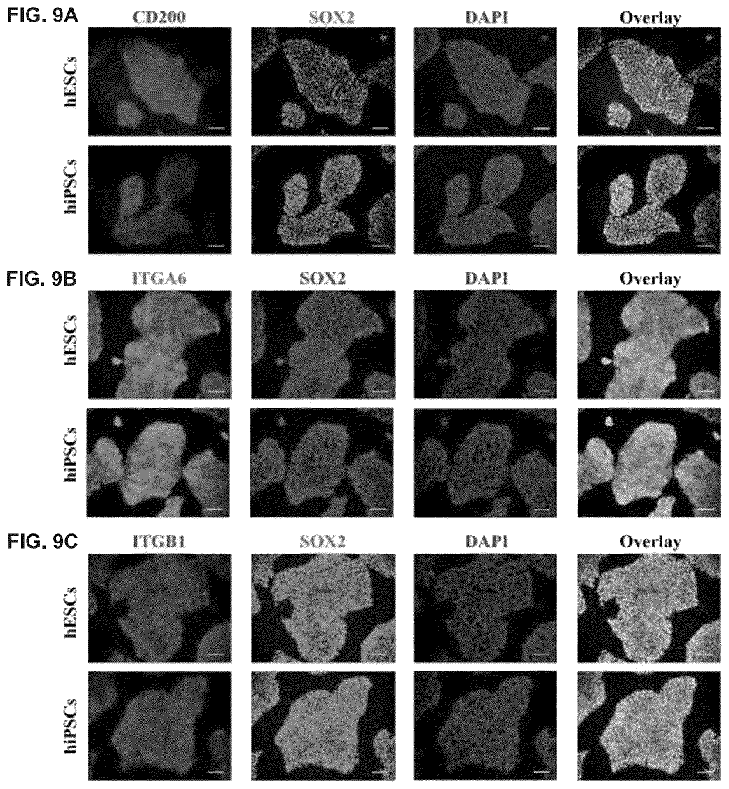

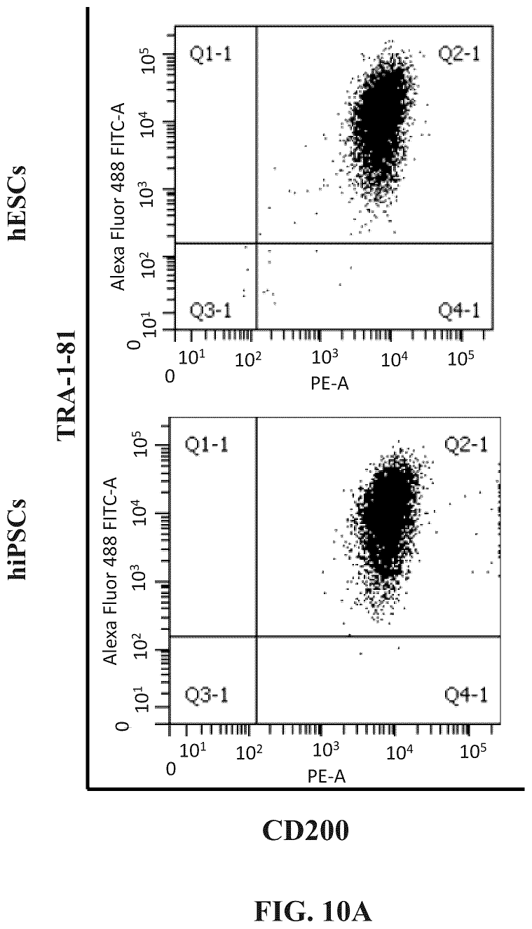

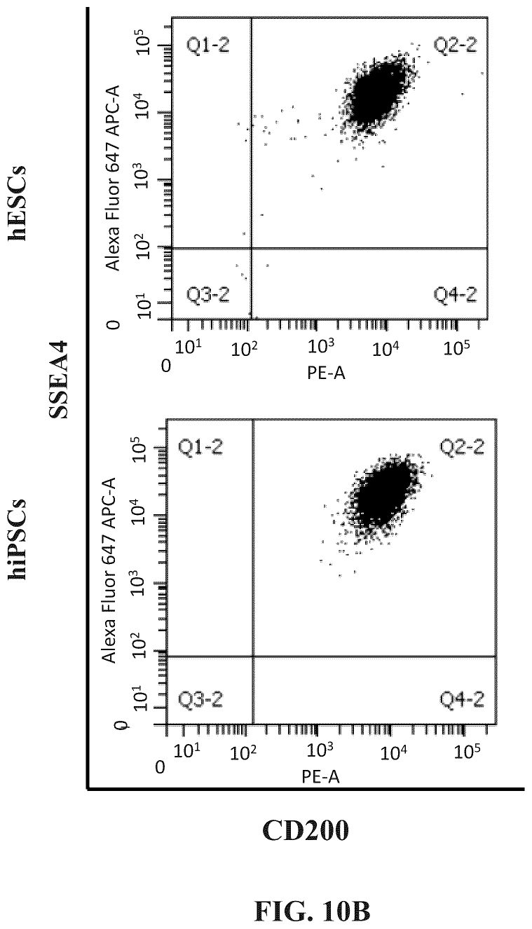

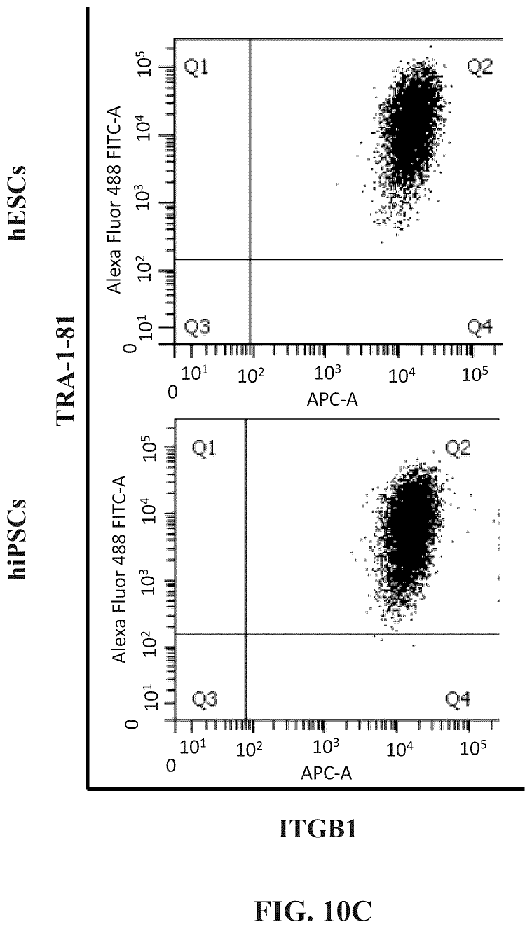

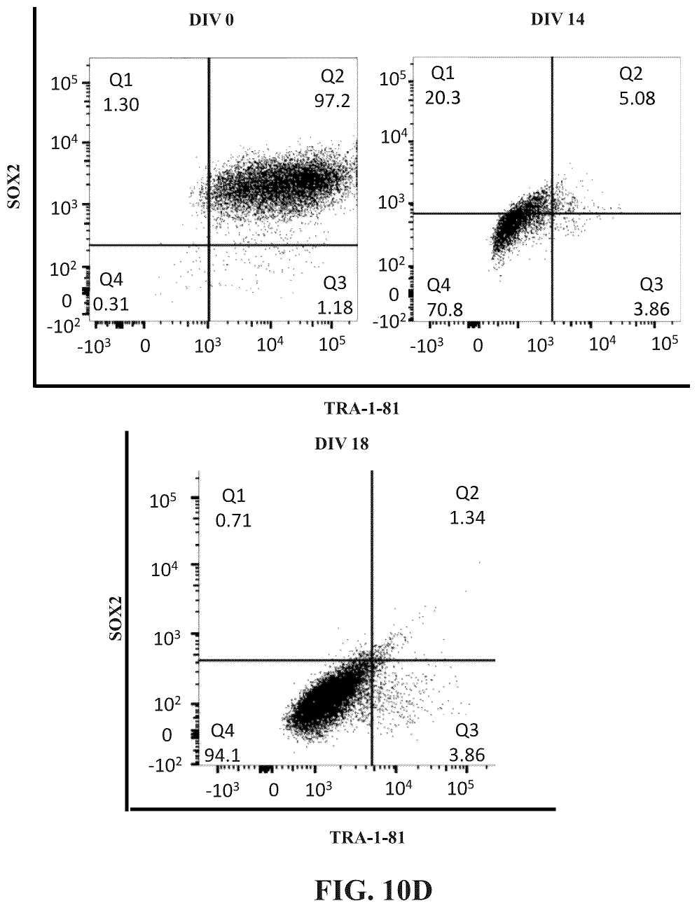

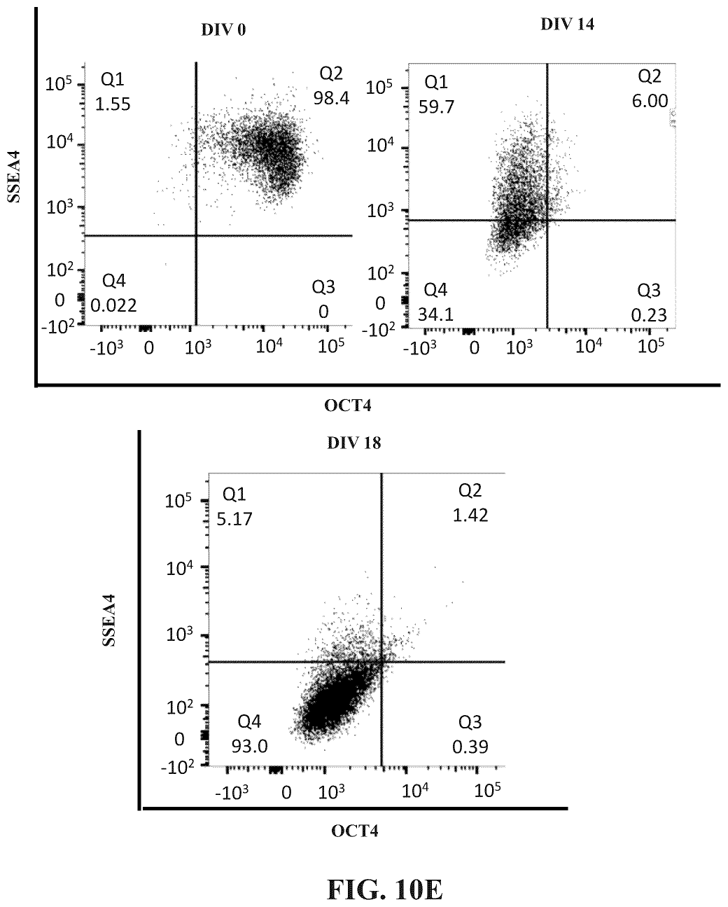

With the conversion of hiPSCs into floating EBs (FIGS. 1A-1C), and the addition of ATRA and L-AA to induce the formation of ectoderm, their expression of classical pluripotency markers began to recede. However, as a bellwether of their ability to ultimately differentiate into HFBSCs, the hiPSCs also expressed CD200, ITGA6, and ITGB1 on their surface—as confirmed by ICC (FIG. 2. Panels A-C; and FIG. 9) and FC (FIG. 2, Panels D-F; and FIGS. 10A-10C)—while still co-expressing pluripotency markers (FIGS. 2, 9, and 10A-10C). As detailed in the next section, these HFBSC-associated markers persisted while the pluripotency markers continued to ebb.

As shown in FIG. 2, co-expression of CD200, ITGA6, and ITGB1 along with cardinal pluripotency markers on hESCs and hiPSCs can be analyzed. Immuno-cytochemical (ICC) analysis is shown in FIG. 2 Panel A for CD200 and NANOG; in FIG. 2 Panel B for ITGA6 and NANOG; in FIG. 2 Panel C for ITGB1 and NANOG. Nuclei of all cells stained blue with DAPI. (Scale bar, 30 mm). Flow cytometric (FC) analysis is shown in FIG. 2 Panel D for CD200 and TRA-1-60; in FIG. 2 Panel E for ITGA6 and SSEA4; and in FIG. 2 Panel F for ITGB1 and TRA-1-60.

Co-expression of CD200, ITGA6, and ITGB1 along with SOX2 on hESCs as well as on hiPSCs, in FIGS. 9A-9C, respectively, using immunocytochemistry. FIG. 9A displays co-expression of CD200 along with SOX2 and DAPI on hESCs and on hiPSCs using immunocytochemistry. FIG. 9B displays co-expression of ITGA6 along with SOX2 and DAPI on hESCs and on hiPSCs using immunocytochemistry. FIG. 9C displays co-expression of ITGB1 along with SOX2 and DAPI on hESCs and on hiPSCs using immunocytochemistry. FIG. 10A depicts flow cytometrical (FC) analysis of markers on hPSC's co-expression of CD200 and TRA-1-81 by hPSC. FIG. 10B depicts flow cytometrical (FC) analysis of markers on hPSC's co-expression of CD200 and SSEA4 by hPSC. FIG. 10C depicts flow cytometrical (FC) analysis of markers on hPSC's co-expression of ITGB1 and TRA-1-81 by hPSC. FIG. 10D depicts flow cytometrical (FC) analysis of down-regulation of pluripotency-associated markers TRA-1-81 on hiPSC-HFBSC at different stages of differentiation. FIG. 10E depicts flow cytometrical (FC) analysis of down-regulation of pluripotency-associated markers OCT4 and SSEA4 at different stages of differentiation.

By DIV 5, the EBs acquired a cystic morphology, at which time they were plated (FIG. 1 Panel D). One day after plating, cells began to migrate out from the plated EBs (FIG. 1 Panel E). The addition of BMP-4 precluded the further differentiation of ectoderm into neuroectoderm. EGF enabled growth. Epithelial colonies, which proved to be HFBSCs (as detailed in the next section), started to appear by DIV 11 and persisted until DIV 18 (FIG. 1 Panel F); keratinocytes appeared on DIV 25 (FIG. 1 Panel G). HFBSCs were harvested no later than DIV 18. Beyond that point, non-engraftable and non-HF-generating keratinocytes would start to emerge.

Expression Dynamics of the Molecules Determining hiPSC-HFBSC Fate

The hPSC-derived cells obtained by the differentiation protocol can be divided into 3 stages: hPSCs (Stage #1) becoming HFBSCs (Stage #2), and then, if no additional cues are presented (including cues from an in vivo environment following transplantation), progression to becoming mature keratinocytes (Stage #3).

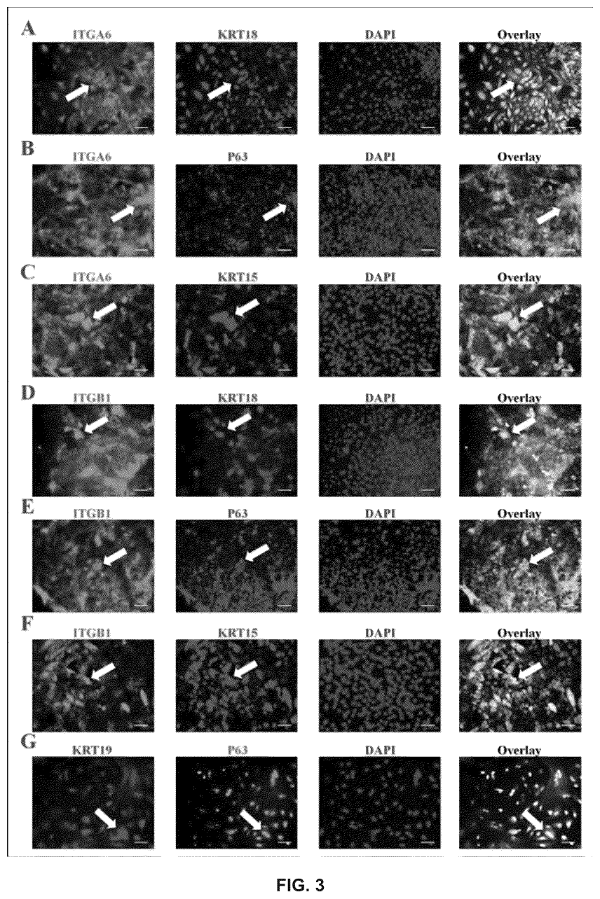

Emergence of the expression of KRT8, KRT18, P63, KRT15, and KRT19 indicated differentiation of the hPSCs toward HFBSCs (see FIG. 3). Critical for consummation of HFBSC generation was the continued co-expression in these cells of integrins a6 and b1 and the surface glycoprotein CD200 (FIG. 3 Panels A-F).

FIG. 3 shows immunocytochemical characterization of hiPSC-HFBSCs. hiPSC-HFBSCs, as generated by the protocol in FIG. 1. The cells were immunoreactive for ITGA6 and KRT18 in FIG. 3 Panel A, for ITGA6 and P63 in FIG. 3 Panel B, for ITGA6 and KRT15 in FIG. 3 Panel C, for ITGB1 and KRT18 in FIG. 3 Panel D, for ITGB1 and P63 in FIG. 3 Panel E, for ITGB1 and KRT15 in FIG. 3 Panel F, and for KRT19 and P63 in FIG. 3 Panel G. Blue nuclear staining with DAPI is used to show all cells in the field. White arrows indicate dual positive cells.

The dynamic changes in these various lineage-determining molecules are detailed based on ICC, FC, and q-PCR.

By ICC and FC, expression of the pluripotency markers that defined the hPSCs decreased (See FIGS. 10D, 10E) concomitant with the emergence of HFBSCs markers (FIG. 3). This was confirmed by RT-PCR; compared to their relative expressions on DIV 0, the expression of OCT4 decreased significantly at DIV 11-18 while the expression of KRT15, KRT19, KRT8, and KRT18 increased significantly at DIV 11-18. These changes heralded the transition from hiPSCs (Stage #1) to hiPSC-HFBSCs (Stage #2). After DIV 18, the cells continued to transition: Compared to their expression on DIV 18, the expression of KRT8 (P<0.01), KRT18 (P<0.01), and KRT15 (P<0.01) decreased significantly by DIV 25, while the expression of KRT5 and KRT14 (keratinocyte markers) increased significantly at DIV 25 (P<0.01), indicating that the cells had moved past Stage #2 into Stage #3, that of mature and potentially terminally differentiated keratinocytes, a cell type which cannot yield HFs (FIG. 4).

FIG. 4 shows the analysis of the dynamics of the relative temporal expression of molecules associated with pluripotency (OCT4), with HFBSC (P63, KRT15, KRT19, KRT8, KRT18), and with keratinocytes (KRT5, KRT14) in hiPSC-derived cells at various days of the differentiation protocol in FIG. 1. Using RT-PCR, we observed that the expression of OCT4 decreased significantly by DIV 11 and was barely detectable by DIV 18 and DIV 25 compared with its expression at DIV 0. Conversely, the expression of KRT15, KRT19, KRT8, and KRT18 increased significantly at DIV 11-18 compared with their expression at DIV 0. The expression of KRT8, KRT18, and KRT15 decreased significantly at DIV 25 compared with their expression at DIV 18 (P<0.01) while the expression of the keratinocyte markers KRT5 and KRT14 increased significantly at DIV 25 compared with their expression at DIV 0 indicating terminal differentiation (P<0.01). Data shown are mean+SD of gene expression from three independent experiments. One-Way ANOVA was used to calculate the P values. *P<0.05; **P<0.01.

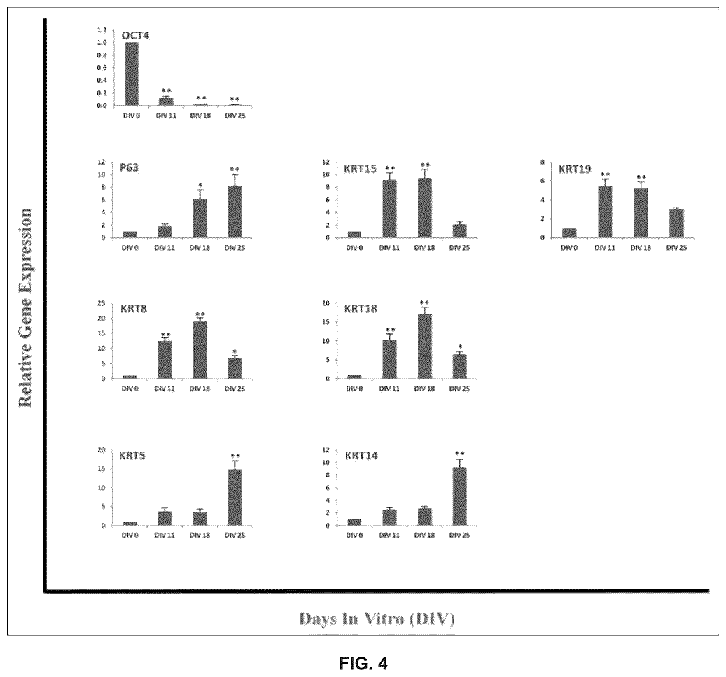

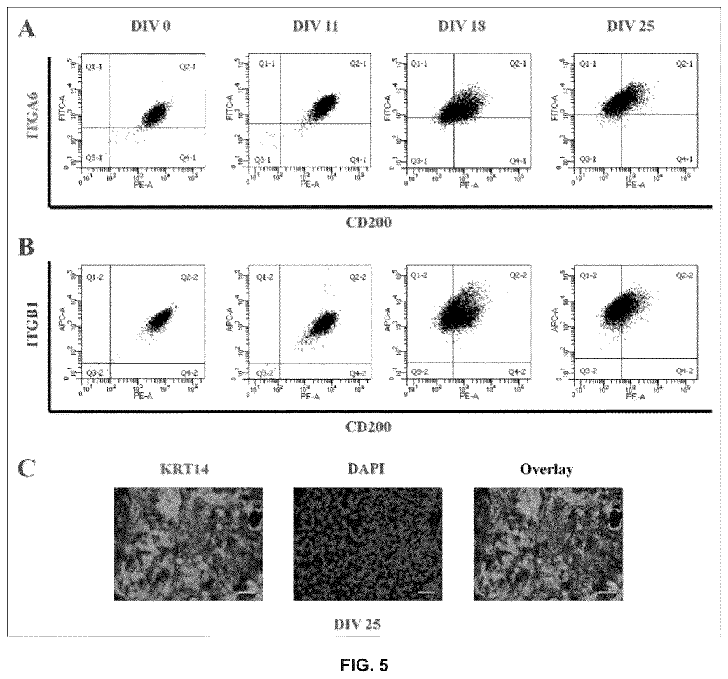

With regard to the surface glycoprotein CD200, its peak expression appeared obligatory for HFBSC generation. CD200 was uniformly expressed by undifferentiated hPSC and continued to be expressed during ectodermal differentiation and differentiation of those ectodermal cells into HFBSCs until DIV 18. CD200 expression persisted even as that of the pluripotency markers disappeared during the earliest differentiation stages. However, starting at DIV 18 and reaching a nadir at DIV 25 (FIG. Panels A and B), CD200 began to downregulate (about 40% of cells have lost CD200 expression by DIV 25) concomitant with increased expression of KRT14 (FIG. 5 Panel C), again indicating that the differentiating cells had “cascaded” through and past the HFBSC state (Stage #2) and had entered the state of being mature terminally differentiated keratinocytes (Stage #3), a non HF-generating cell type.

FIG. 5 shows the characterization of the hiPSC-derived keratinocytes in relation to the hiPSC-HFBSCs which emerge earlier. Flow cytometric (FC) analysis showing co-expression of CD200 and ITGA6 in FIG. 5 Panel A and CD200 and ITGB1 in FIG. 5 Panel B on hiPSCs on DIV 0-25. In the analyses shown, the upper right quadrant contains cells positive for both ITGA6 and CD200 or ITGB1 and CD200, respectively. The upper left quadrant contains cells that are positive only for ITGA6 or ITGB1. The lower right quadrant contains cells that are positive only for CD200. The lower left quadrant contains double-negative cells. Cells in the upper half are ITGA6+ or ITGB1+ while cells in the lower half are ITGA6− or ITGB1−. Cells on the right side are CD200+ while those on the left side are CD200−. On DIV 0, all of the cells are present at the upper right quadrant, indicating that all of the cells express both ITGA6 and CD200 as well as ITGB1 and CD200. ITGA6 and ITGB1 continue to be expressed on hiPSC-HFBSCs and keratinocytes (i.e., are in the upper right quadrant) from DIV 0 to DIV 25. On DIV18 one can see about 25% of the cells moving from the upper right quadrant to the upper left quadrant, that is, starting to lose CD200 expression. By DIV 25, more cells have lost CD200 expression and moved from the upper right quadrant to the upper left quadrant (about 40% of the cells); CD200 expression reaches its nadir at this point (DIV 25). It is those CD200− cells that become KRT14+ and KRT5+ mature differentiated keratinocytes on DIV 25 (as illustrated in FIG. 5 Panel C). Immunocytochemical analysis in FIG. 5 Panel C shows expression of KRT14 at DIV 25 of the differentiation protocol (the point at which CD200 expression has ebbed) indicating the emergence of mature keratinocytes (Stage #3 cells), a point beyond the HFBSC stage (Stage #2 cells) and one that cannot yield HF generation in vivo following transplantation. Blue nuclear staining with DAPI shows all cells in the field. (Scale bar, 30 mm).

Hence, downregulation of CD200 with concomitant upregulation of KRT14 appeared to separate the HFBSC stage (Stage #2) from the keratinocyte stage (Stage #3). This “border zone” may hold translational significance if Stage #2 cells (hiPSC-HFBSCs) but not Stage 3 cells (KRT14+ keratinocytes), could engraft to yield HFs in vivo.

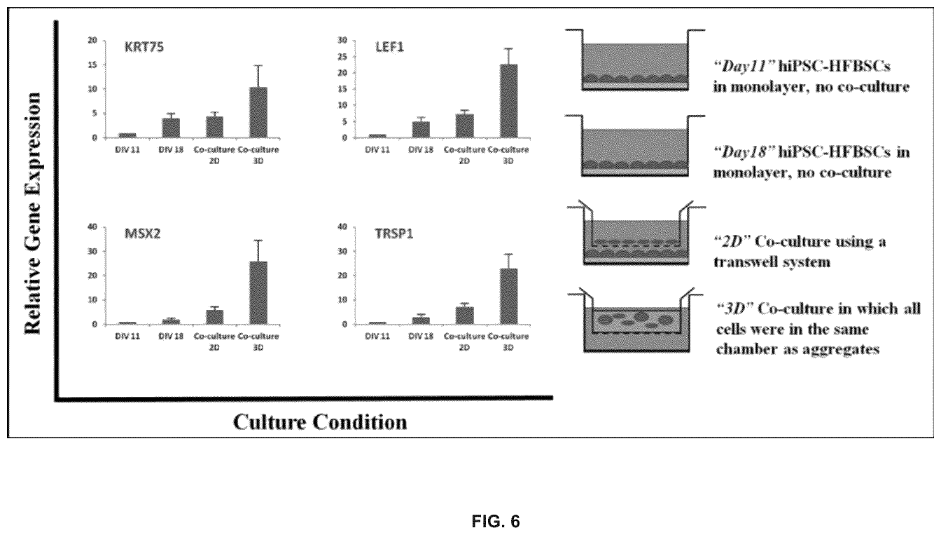

Before testing the above rationale/hypothesis directly via transplantation studies, one may need to determine whether Stage 2 cells had the competence to express hair differentiation markers when exposed to proper inductive cues (FIG. 6).

FIG. 6 shows the conditions for the co-culture of hiPSC-HFBSCs and MDCs. Marked upregulation of hair-related gene expression in hiPSC-HFBSCs following their induction by trichogenic mesenchymal cells via cell-cell contact in 3D co-cultures is compared with hiPSC-HFBSC cells alone in monolayer (at DIV 11 or DIV 18), or co-cultures in a transwell system (which allowed interaction between hiPSC-HFBSC cells and mesenchymal cells via diffusible factors alone, the cells separated by a porous membrane that permitted passage of molecules but not cells.) Data shown are mean+SD of gene expression from three independent experiments.

The “bulge activation hypothesis” holds that signals from the dermal papillae (DP) (the mesenchymal component of the HF) stimulate resting HFBSCs to generate transient amplifying cells which can then form HFs and hair shafts in vivo (Catsarelis, G. et al. Label-retaining cells reside in the bulge area of pilosebaceous unit: implications for follicular stem cells, hair cycle, and skin carcinogenesis. Cell. 1990; 61(7):1329-1337). Accordingly, an experiment was conducted to co-culture hiPSC-HFBSCs with freshly isolated MDCs for 1 week using two different paradigms (FIG. 6). In the first, a transwell system was employed in which hiPSC-HFBSCs were placed as a monolayer in the bottom well while an equal number of MDCs were seeded as a monolayer onto porous inserts in the upper well; the pores were a size that would allow the passage of only molecules but not cells. This system can determine whether the Stage 2 cells in monolayer (2 dimensions [2D]) would respond to diffusible signals. In the second system, equal numbers of the two cell types (hiPSC-HFBSCs and MDCs) were co-cultured in a 3 dimensional (3D) sphere (in which all cells were in the same top chamber as aggregates) allowing for cell-cell contact. qPCR analysis for hair differentiation markers was then performed on the co-cultured hiPSC-HFBSCs (Stage 2 cells) on DIV 18. Not only was HF-associated gene expression in the two co-culture systems compared with each other but also with expression by the Stage 2 cells cultured alone in monolayer on DIV 11 and DIV 18. Expression of the HF-associated genes KRT75, MSX2, LEF1 and TRPS1 showed a marked increase in the 3D co-culture system, much greater than that in the 2D transwell system or in the Stage 2 cells alone on DIV 11 and DIV 18 (FIG. 6). Stated another way, Stage 2 cells exposed to diffusible factors alone for induction (i.e., via transwell) showed an expression of HF-associated genes that was slightly higher than if the cells had been cultured and matured alone in monolayer. These data support the likelihood of successful HF generation following engraftment of Stage 2 cells and demonstrate the necessity for an in vivo environment with cell-contact in a proper 3D niche to achieve full induction. The competence of Stage 2 cells can be missed without transplantation.

Characterization In Vivo of hiPSC-HFBSCs Following Transplantation

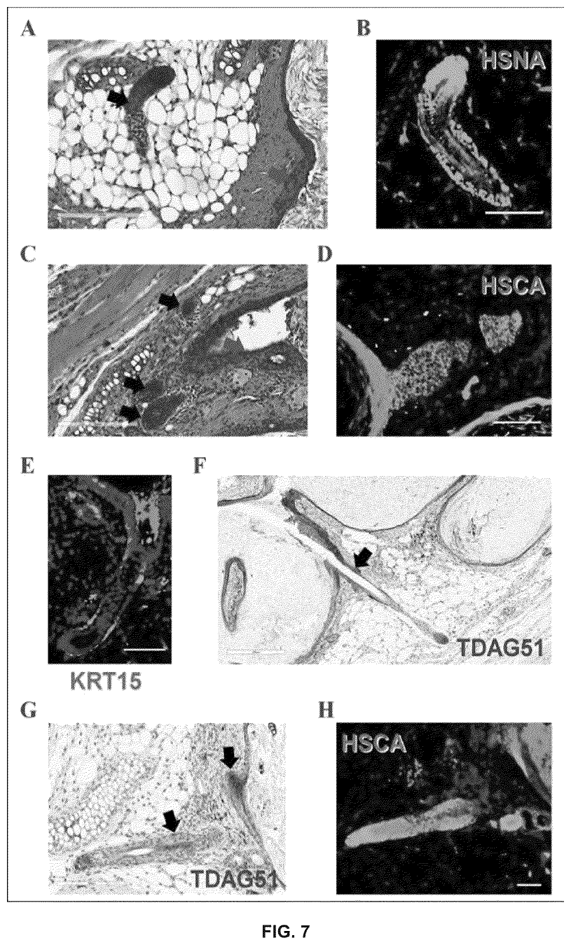

In search for the optimal molecular profile for inducing hiPSCs to yield HF-competent HFBSCs, sustained co-expression of CD200, ITGA6 and ITGB1 together with emergence of other HFBSC-associated gene products such as P63, KRT15, KRT19, and KRT18 (i.e., Stage 2 cells) can be a solution. Towards that end, hiPSC-HFBSCs were translated into SCID mice at DIV 16. DIV 16 represented the time point at which there was maximal expression of CD200 (before its down-regulation, as shown by FC) as well as of KRT15, KRT19, and KRT18 (as shown by RT-PCR). Transplantation of the hiPSC-HFBSCs was accomplished by co-injection of HFBSCs and MDCs intradermally above the muscle coat, where the cells could remain tightly packed and in contact with each other with little dispersion. The hiPSC-derived HFBSCs yielded HF in vivo (FIG. 7). On the other hand, transplantation of cells at DIV 25, after downregulation of CD200, failed to generate HFs in vivo. In other words, mature keratinocytes, Stage 3 cells, were not competent to generate HFs in vivo. As suggested by the above-described 3D co-culture experiments, cell aggregation and cell-cell interaction were pivotal for the Stage 2 cells to participate in organogenesis and yield HFs. Highly dispersed grafts injected in the subcutaneous fat did not develop HF, nor did injection of MDCs alone.

FIG. 7 shows histologic evaluation of donor-derived HFs following intradermal transplantation of hiPSCs-HFBSCs. Injection of CD200+/ITGA6+/ITGB1+ hiPSC-derivatives intradermally into SCID mice (above the muscle coat such that they can maintain cell-cell contact with little dispersion) resulting in small epidermal cysts with HFs radiating from them, proof of HFBSC differentiation and HF generation competence. FIG. 7 Panel A shows a representative HF (black arrow) and epidermal cyst lined by multilayered epidermis (red arrow). FIG. 7 Panel B shows positive immunoreactivity of this HF for HSNA (green). FIG. 7C shows a small epidermal cyst showing a multi-layered epidermis (red arrow) with multiple HFs radiating from it (black arrows). FIG. 7 Panel D shows positive immunoreactivity for HSCA (green) in the reconstituted epidermis and HF. FIG. 7 Panel E shows positive immunostaining (green) of a representative reconstituted HF using an antibody against KRT15 (a known HFBSC stem cell marker); the immunopositive cells are present in the basal layer of epidermis, in the bulge region, and in the basal layer of the outer root sheath (red arrows).

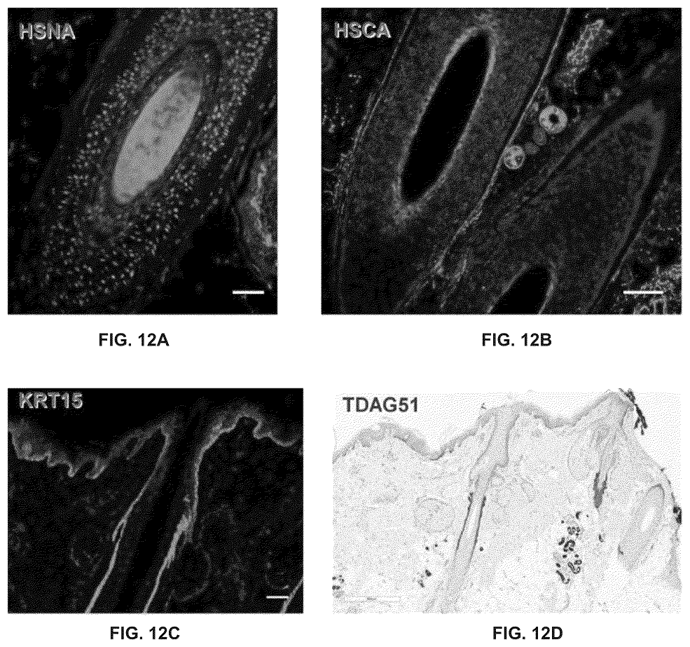

Positive immunoperoxidase staining (brown) of representative reconstituted HFs with an antibody against TDAG51 (a known HF stem cell marker) are shown in FIG. 7. The stained cells are present in the bulge region (black arrows) in FIG. 7 Panels F and G. According to FIG. 7 Panel H the reconstituted epidermis and HF in (G) is immunopositive for HSCA (green). (Scale bar, 100 mm). (see FIGS. 12 and 13 for additional immunohistochemistry supporting human origin of the HFs).

Three to six weeks following transplantation, histological analysis showed that the injected Stage 2 cells aggregated to form small cystic spheres in the host dermis. Human-like multilayered epidermis was also formed in the grafts. The cysts consisted of both basal keratinocytes and stratified epidermis. Pilosebaceous units growing outward from the cyst were evident (FIG. 7). In FIG. 7, one can see the morphology of the hiPSC-derived HFs, including their discrete component parts (e.g., the hair shaft, the hair matrix, the outer root sheath.). Although the new follicles in this system do not usually produce skin surface hair shafts (because the new follicle growth occurs in the deep dermis), we expected that, if the trichogenic cells were implanted superficially enough, the hair shafts would egress, individually or in tufts. Indeed, the hiPSC-HFBSCs not only reconstituted the epithelial components of the HF but also the interfollicular epidermis. These findings indicated that hiPSC-derived HFBSC were capable of generating epidermis and that they responded to inductive dermal signals in vivo in the developmentally appropriate niche to generate HFs.

Human origin of the epithelial cells in the HFs and epidermis was confirmed by immunopositivity for the human-specific nuclear antigen (HSNA) which was detected in about 60-70% of growing HFs cells. Human derivation of the cells was further confirmed by the positive immunoreactivity for a human-specific cytoplasmic antigen (HSCA) (FIGS. 7B, 7D, 7H; 11, and 12). Human cells were detected in the generated HFs for at least 6 weeks following transplantation (when the experiments were terminated). Hence, most of the cells comprising the HFs arise from the implanted human-origin cells. That 30-40% of the HF cells were of non-human host origin suggest that, in the context of transplantation, endogenous cells also contributed to the epithelial and dermal lineages. The HFs formed in our system do not make contact with host epithelium (neither skin appendages nor epidermis); therefore the host contribution to the epithelium came from the surrounding mesenchyme or circulating cells, a process that implied a mesenchymal-to-epithelial transition (see, e.g., Zeisberg, M. et al. BMP-7 counteracts TGF-beta1-induced epithelial-to-mesenchymal transition and reverses chronic renal injury. Nat Med. 2003; 9(7):964-968). This observation was consistent with earlier reports that either vicinal (see McElwee, K. J. et al. Cultured peribulbar dermal sheath cells can induce hair follicle development and contribute to the dermal sheath and dermal papilla. J Invest Dermatol. 2003; 121(6):1267-1275) or bone marrow-derived circulating cells (see Kataoka, K. et al. Participation of adult mouse bone marrow cells in reconstitution of skin. Am J Pathol. 2003; 163(4): 1227-1231) will incorporate into regenerating skin and hair, but suggests that transplantation (including of human cells) can evoke that response from the host.

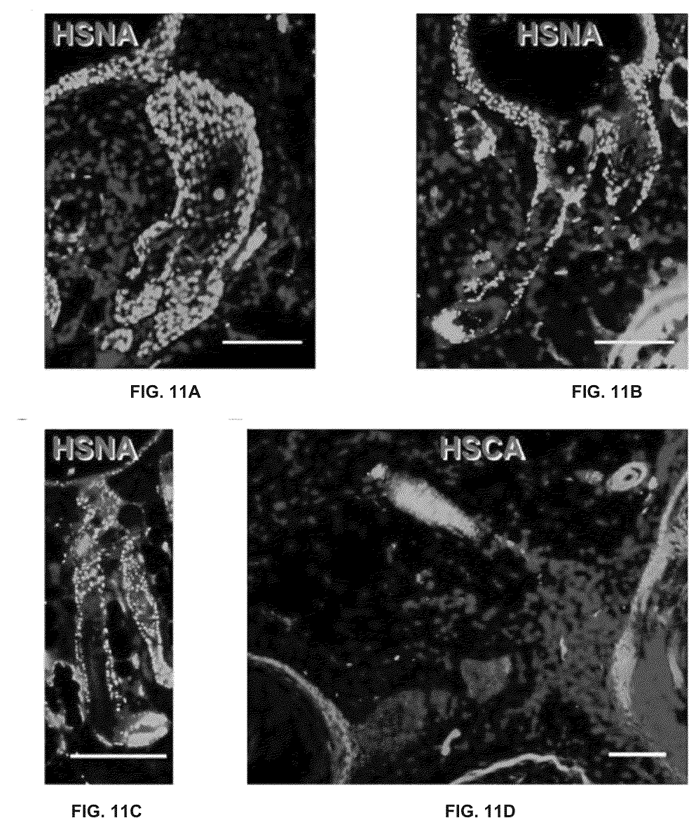

FIGS. 11A-11C show human origin of the donor-derived reconstituted HFs by showing the positive immunoreactivity for human specific nuclear antigen (HSNA)(green). FIG. 11D shows human origin of the donor-derived reconstituted HFs by showing the positive immunoreactivity for human specific cytoplasmic antigen (HSCA)(green) in the reconstituted epidermis and HF.

FIG. 12A displays that a primary human HF can serve as a positive control for human specific nuclear antigen (HSNA) (green). FIG. 12B displays that a primary human HF can serve as a positive control for human specific cytoplasmic antigen (HSCA) (green). FIG. 12C displays that a primary human HF can serve as a positive control for KRT15 (green). FIG. 12D displays that a primary human HF can serve as a positive control for TDAG51.

A cell-based treatment for alopecia has a challenge to ascertain that newly generated HFs contain a mechanism for cycling. The donor-derived HFs bore a “bulge region” in vivo rendering them capable of cycling and renewal. Indeed, KRT15 expression in the bulge region of the chimeric HFs and in the basal layer of the epidermis (FIG. 7 Panel E) were found. The expression of T-Cell Death-Associated Gene 51 (TDAG51) (also called PHLDA1, Pleckstrin Homology Like Domain Family A Member 1), a HF stem cell marker (see, e.g., Sellheyer, K. et al. PHLDA1 (TDAG51) is a follicular stem cell marker and differentiates between morphoeic basal cell carcinoma and desmoplastic trichoepithelioma. Br J Dermatol. 2011; 164(1): 141-147), further confirmed the presence of HFBSCs in the bulge region of the donor-derived HFs (FIGS. 7F, 7G). Never noted were neoplastic cells, cells inappropriate to the dermis, or cell overgrowth or deformation.

Assessing the Necessary & Sufficient Elements for HF Generation

An important step in confirming the necessity and causality of the putative suite of developmental determinants was to first eliminate and then re-add each factor systematically and demonstrate first an inability and then a reacquisition of the ability to yield engraftable HF-generating HFBSCs. However, all of the seven molecules (markers) required for complete HF generation were also fundamental to the earliest stages of normal development. Neither of the seven genes can be knock-out, and certainly not all seven, (even conditionally) in the hPSCs without disrupting normal development and confounding interpretation of the experiments. Furthermore, they could be knocked out even in the starting fibroblasts prior to being reprogrammed into hiPSCs because each is obligatory for the reprogramming process itself as well as for subsequent expansion, self-renewal, and acquisition of pluripotency (see, e.g., Sellheyer, K. et al. PHLDA1 (TDAG51) is a follicular stem cell marker and differentiates between morphoeic basal cell carcinoma and desmoplastic trichoepithelioma. Br J Dermatol. 2011; 164(1):141-147; and Sellbeyer, K. et al. Follicular stem cell marker PHLDA1 (TDAG5 1) is superior to cytokeratin-20 in differentiating between trichoepithelioma and basal cell carcinoma in small biopsy specimens. J Cutan Pathol. 2011; 38(7):542-550). Given these limitations, a series of studies that would serve the same purpose as a “knock-out” but without perturbing the system and obfuscating interpretation through ambiguity, were performed, as detailed below.

The expression profile of each of the markers that hPSC-derived HFBSC ultimately achieve is mapped. Test transplants of cells were done at each epoch along the developmental trajectory from “epiblast” (i.e., undifferentiated hPSC) to “ectoderm” to “HFBSC” to “keratinocyte”. Each period had a different constellation of markers among the seven. The question is which constellation yielded HFs in vivo.

Similar to what has been observed above, P63, KRT15, KRT18, and KRT19 start to be expressed at DIV 11, peak at DIV18, and decrease by DIV 25. CD200, while expressed earlier than DIV 11 (when the cells are still in their pluripotent state), ebb by DIV 25, the point at which KRT5 and KRT14 expression becomes ascendant, heralding the emergence of keratinocytes. ITGA6 and ITGB1 are expressed starting at DIV 0 through DIV 25; if these integrins are not expressed, HF also fails to develop. Transplantation of cells prior to DIV 11 or after DIV 25 fails to yield HF. Therefore, we could conclude that HF generation requires the expression of CD200, ITGA6, ITGB1, KRT15, KRT18, KRT19, and P63 and that a constellation lacking one or more of these molecules was insufficient to yield HFs.

Therefore, transplantation at DIV 16-18 may be favored, at which time the cells have lost expression of pluripotency markers, continue to express integrins ITGA6 and ITGB1, have attained optimal expression levels of the HFBSC markers KRT15 and KRT19 (in conjunction with P63), express the epithelial marker KRT18, have not yet experienced downregulation of the surface glycoprotein CD200, have not yet started expressing the keratinocyte-associated molecules KRT5 and KRT14, and have attained optimal proliferative capacity to allow a well-populated graft. The optimal site for transplantation was intradermal and above the muscle coat to prevent cell dispersion. Transplantation of MDCs alone failed to yield HFs in vivo.

Analysis

To find out how best to produce and select pluripotent stem cell derivatives for reliable and efficient therapeutic HF replacement, a developmental approach to this question can be taken by generating HFs as they might emerge if starting in the epiblast (a stage emulated by hESCs and, for clinical use, patient-specific hiPSCs) and progressing iteratively through gastrulation and on to dermal organogenesis.

The dynamic up- and down-regulation of specific molecules that serve as landmarks for each stage of this developmental process can be mapped. From the mapped dynamic, one can find the precise point at which the appropriate molecular and developmental requirements were attained by the cell (as indicated by surrogate biomarkers) to yield optimal HF generation in vivo if transplanted. What emerged was a 3-stage profile that entailed differentiating hiPSCs (Stage #1) toward becoming keratinocytes in vitro (Stage 3) but, just prior to that endpoint, capturing and isolating an intermediate transient progenitor cell population (Stage #2), which are bona fide HFBSCs, not only based on their co-expression of HFBSC-associated molecules but, most importantly, by their ability to generate HFs in vivo upon transplantation. Cells that cascaded beyond “HFBSC Stage #2” into “keratinocyte Stage #3” lost that ability to generate HFs upon grafting. In short, to ensure that hiPSC-derived HFBSC have acquired the competence for HF generation, they must come to express, at the time of transplantation, CD200, ITGA, and ITGB1 on their surface, and KRT18, P63, KRT15, and KRT196, but not KRT5 or KRT14 (keratinocyte markers), intracellularly. Expression of the HFBSCs markers P63, KRT15, and KRT19 as well as the HFBSC-associated epithelial marker KRT18 increases significantly from DIV 11 until DIV 18 (marking the transition from hiPSC Stage #1 to HFBSC Stage #2. Although CD200 is expressed starting in Stage #1, it begins to wane at DIV 18, reaching its nadir at DIV 25. Keratinocyte markers KRT5 and KRT14 are low through Stages #1 and #2, but dominate by DIV 25. DIV 18 appears to represent a developmental border between Stage #2 (CD200 engraftable HF-yielding HFBSCs) and Stage #3 (unengraftable mature non-HF yielding KRT5/14 keratinocytes). Therefore, to yield HFs, transplantation, we learned, should take place after DIV 11 but no later than DIV 18; we have chosen DIV 16-18 as our optimal transplant time to insure that all pluripotency markers have downregulated and the HFBSC-associated molecules are at their peaks.

The expression of CD200, ITGA6 and ITGB1 on hiPSCs and hESCs is a sine qua non for the differentiation of hPSCs into HFBSCs is unexpected. The ICC and FC data not only clearly confirm their expression on Stage #1 cells, in association with other known pluripotency markers such as NANOG, SOX2, TRA-1-60, TRA-1-81, and SSEA4, but suggest that in the absence of such integrin-related molecules and glycoproteins, hPSCs cannot proceed to becoming HFBSC Stage #2 cells with competence for yielding HFs. There are two supporting evidences. First, the 3D co-cultures show that induction of the molecules mediating HF generation required cell-cell contact between receptive HFBSCs and trichogenic mesenchymal cells, an interaction mediated by these integrins. Second, downregulation of CD200 coincided with progression of Stage #2 HFBSCs toward a Stage #3 keratinocyte fate incapable of HF generation, while transplantation of the Stage #2 cells at the peak of their CD200 expression in conjunction with expression of the integrins consistently yielded HFs in vivo.

Beyond our empiric data, the expression of these surface glycoproteins makes biologic sense for creating hPSC-derived HFs. Integrins are transmembrane glycoproteins composed of an x subunit and a 13 subunit that are linked via non-covalent bonds. The x6 subunit associates with the 131 subunit or the 134 subunit to form x6131 and x6134 integrin heterodimers. x6131 is expressed on a variety of cell types and functions as a cellular receptor for matrix laminin42. ITGA6 is the x6 subunit (also known as CD49f); ITGB1 is the 131 subunit. x6131 integrins along with x6134 integrins confer functional characteristics to stem cells; x6131 may be the dominant integrin. More than 30 different stem cell types have been found to express ITGA6 on their membranes. During the reprogramming of fibroblasts into hiPSCs, ITGA6 is upregulated and focal adhesion kinase (FAK) is inactivated (via dephosphorylation). During differentiation of the hiPSCs, the converse takes place: ITGA6 levels diminish and FAK is activated (phosphorylated at residue Y397). Activation of ITGB1 also leads to FAK phosphorylation and reduction of NANOG, OCT4, and SOX2. Knockdown of ITGA6 can mimic ITGB1 activation and reduce or eliminate normal hESC and hiPSC colony development, self-renewal, and pluripotency. Hence, while the presence of these two arms of the integrin system are necessary for hPSC maintenance and differentiation, they may play dynamic opposing roles; obviously an equilibrium must be struck between them for first reprogramming and then development to proceed—in this case, all the way to becoming HF-competent HFBSCs. CD200 is a glycoprotein widely expressed on cancer stem cells (breast, colon, hematopoietic) and may be required to maintain growth, self-renew, metastasis, and to evade the immune system38. It is also a marker of HFBSCs (as well as limbal stem cells). This combination of surface molecules likely interacts with specific matrix receptors during culture which, in turn, influences the hiPSCs' adhesion, survival, proliferation, and entrance into particular differentiation “programs”. The expression of these molecules on hiPSCs appeared to ensure that the cells had acquired the molecular competence for becoming HFs in vivo. In fact, the necessity of these surface markers may be used for cytometry to extract hiPSC derivatives in Stage #2 that can yield HFs from a heterogeneous population that might contain cells disadvantageous for this therapeutic indication. For regulatory purposes, it might be a way to isolate therapeutic Stage #2 cells from undifferentiated and potentially tumorigenic hPSCs. These same markers, which are avatars for a desirable developmental trajectory, may be used to identify drugs that might enhance the efficiency of HF generation.

Based on the developmental dynamics mapped above, the transplantation at DIV 16-18 is favored. At DIV 16-18 the cells have lost expression of pluripotency markers, continue to express integrins ITGA6 and ITGB1, have attained optimal expression levels of the HFBSC-associated markers KRT15, KRT18, and KRT19 (as well as P63), have not yet experienced downregulation of CD200, have not yet started expressing the keratinocyte-associated molecules KRT5 and KRT14, and have attained optimal proliferative capacity to allow a well-populated graft. Transplantation should be done intradermally above the muscle coat, where the cells can remain tightly packed and in contact with each other with little dispersion. The guidance provided by the present study may bring us closer to replacing HFs lost in non-cicatricial and cicatricial alopecia through the use of a patient's own hiPSC-derived follicular stem cells.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A method of growing a hair follicle comprising:
(a) preparing human induced pluripotent stem cells (hiPSCs);
(b) differentiating the hiPSCs into hair follicle bulge stem cells (HFBSCs); and
(c) implanting the HFBSCs into skin of a subject, wherein the subject is a human subject.

2. The method of claim 1, wherein the hiPSCs have one, two, three, four, five, six, or more markers selected from the group consisting of CD200, ITGA6, ITGB1, OCT4, NANOG, SOX2, TRA-1-60, TRA-1-81, and SSEA4.

3. The method of claim 1, wherein the HFBSCs have one, two, three, four, five, six, or seven markers selected from the group consisting of CD200, ITGA6, ITGB1, KRT15, KRT18, KRT19, and P63.

4. The method of claim 1, wherein the preparing in (a) comprises introducing, by electroporation, non-integrating episomal plasmid vectors encoding OCT3/4, SOX2, KLF4, L-MYC, LIN28 and an shRNA for human p531.

5. The method of claim 4, wherein the electroporation is via a Neon transfection system.

6. The method of claim 1, wherein the differentiating in (b) comprises formation of embryoid bodies (EBs) in a floating culture.

7. The method of claim 6, wherein the differentiating in (b) comprises plating the EBs onto coated plates.

8. The method of claim 7, wherein the coated plates are collagen I coated plates.

9. The method of claim 1, further comprising, prior to (c), (b1) differentiating the hiPSCs into keratinocytes.

10. The method of claim 9, wherein the differentiating in (b1) comprises employing all-trans retinoic acid (ATRA) and L-ascorbic acid (L-AA) to induce the hiPSC to form ectoderm and then the addition of bone morphogenic protein-4 (BMP-4) and epidermal growth factor (EGF).

11. The method of claim 10, wherein the differentiating in (b1) is according to a sequential differentiation protocol.

12. The method of claim 1, wherein the implanting in (c) comprises intradermal injection, or the implanting in (c) occurs at 15-19 days in vitro (DIV).

13. (canceled)

14. The method of claim 12, wherein the implanting in (c) occurs 16-18 DIV.

15. The method of claim 1, wherein the HFBSCs have not yet started expressing the keratinocyte associated molecules KRT5 and KRT14.

16. The method of claim 1, further comprising treating hair loss and/or a condition in the subject in need thereof, the condition is alopecia, ectodermal dysplasia, monilethrix, Netherton syndrome, Menkes disease, or hereditary epidermolysis bullosa.

17. (canceled)

18. A composition, comprising (a) hair follicle bulge stem cells (HFBSCs); and (b) media, wherein the HFBSCs express markers CD200, ITGA6, ITGB1, KRT15, KRT18, KRT19, and P63.

19. The composition of claim 18, wherein the HFBSCs do not express the keratinocyte associated molecules KRT5 or KRT14.

20. The composition of claim 19, wherein the composition is made by a process comprising:
(a) preparing human induced pluripotent stem cells (hiPSCs); and
(b) differentiating the hiPSCs into the HFBSCs.

21. The composition of claim 20, wherein the hiPSCs have one, two, three, four, five, six, or more markers selected from the group consisting of CD200, ITGA6, ITGB1, OCT4, NANOG, SOX2, TRA-1-60, TRA-1-81 and SSEA4.

22-26. (canceled)

27. A method for hair follicle replacement, the method comprising:
a. obtaining human pluripotent stem cells (hPSCs), wherein the hPSCs are human induced pluripotent stem cells derived hair follicle bulge stem cells (hiPSC-HFBSC);
b. differentiating the hPSCs, thereby producing differentiated hPSCs toward becoming keratinocytes;
c. capturing and isolating at least a portion of the differentiated hPSCs, wherein the portion of the differentiated hPSCs expresses hair follicle bulge stem cell markers (HFBSCM); and
d. transplanting the portion of the differentiated hPSCs into a patient in need thereof, wherein the patient is a human.

28-46. (canceled)
